SEQUENCE LISTING (27 sequences)

SEQ ID NO: 1 - protein, 452 aa, Homo sapiens
MSTAVLENPGLGRKLSDFGQETSYIEDNCNQNGAISLIFSLKEEVGALAKVLRLFEENDVNLTHIESRPSRLKKDEYEFFTHLDKRSLPALTNIIKILRHDIGATVHELSRDKKKDTVPWFPRTIQELDRFANQILSYGAELDADHPGFKDPVYRARRKQFADIAYNYRHGQPIPRVEYMEEEKKTWGTVFKTLKSLYKTHACYEYNHIFPLLEKYCGFHEDNIPQLEDVSQFLQTCTGFRLRPVAGLLSSRDFLGGLAFRVFHCTQYIRHGSKPMYTPEPDICHELLGHVPLFSDRSFAQFSQEIGLASLGAPDEYIEKLATIYWFTVEFGLCKQGDSIKAYGAGLLSSFGELQYCLSEKPKLLPLELEKTAIQNYTVTEFQPLYYVAESFNDAKEKVRNFAATIPRPFSVRYDPYTQRIEVLDNTQQLKILADSINSEIGILCSALQKIK

SEQ ID NO: 2 - DNA, 218 bp, Artificial Sequence
  Synthetic oligonucleotide
gggggaggctgctggtgaatattaaccaaggtcaccccagttatcggaggagcaaacaggggctaagtccaccgggggaggctgctggtgaatattaaccaaggtcaccccagttatcggaggagcaaacaggggctaagtccaccgggggaggctgctggtgaatattaaccaaggtcaccccagttatcggaggagcaaacaggggctaagtccac

SEQ ID NO: 3 - DNA, 233 bp, Artificial Sequence
  Synthetic Oligonucleotide
aaatgacctattaagaatatttcatagaacgaatgttccgatgctctaatctctctagacaaggttcatatttgtatgggttacttattctctctttgttgactaagtcaataatcagaatcagcaggtttgcagtcagattggcagggataagcagcctagctcaggagaagtgagtataaaagccccaggctgggagcagccatcacagaagtccactcattcttggcagg

SEQ ID NO: 4 - DNA, 77 bp, Artificial Sequence
  Synthetic Oligonucleotide
ctaaggtaagttggcgccgtttaagggatggttggttggtggggtattaatgtttaattaccttttttacaggcctg

SEQ ID NO: 5 - DNA, 248 bp, Artificial Sequence
  Synthetic Oligonucleotide
aatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttagttcttgccacggcggaactcatcgccgcctgccttgcccgctgctggacaggggctcggctgttgggcactgacaattccgtggtgtt

SEQ ID NO: 6 - DNA, 602 bp, Artificial Sequence
  Synthetic Oligonucleotide
gataatcaacctctggattacaaaatttgtgaaagattgactggtattcttaactttgttgctccttttacgctttgtggatacgctgctttattgcctttgtatcttgctattgcttcccgtttggctttcattttctcctccttgtataaatcctggttgctgtctctttttgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcatcggactag

SEQ ID NO: 7 - DNA, 631 bp, Artificial Sequence
  Synthetic Oligonucleotide
accaggttctgttcctgttaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccctggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccatggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcctgtttcgcctcgggctcctcgag

SEQ ID NO: 8 - DNA, 234 bp, Artificial Sequence
  Synthetic Oligonucleotide
cctagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggaa

SEQ ID NO: 9 - DNA, 133 bp, Artificial Sequence
  Synthetic Oligonucleotide
aggaacccctagtgatggagttggccactccctctctgcgcgctcgctcgctcactgaggccgggcgaccaaaggtcgcccgacgcccgggctttgcccgggcggcctcagtgagcgagcgagcgcgcagaga

SEQ ID NO: 10 - DNA, 130 bp, Artificial Sequence
  Synthetic Oligonucleotide
ctgcgcgctcgctcgctcactgaggccgcccgggcaaagcccgggcgtcgggcgacctttggtcgcccggcctcagtgagcgagcgagcgcgcagagagggagtggccaactccatcactaggggttcct

SEQ ID NO: 11 - DNA, 1359 bp, Artificial Sequence
  Synthetic Oligonucleotide
atgtccactgcggtcctggaaaacccaggcttgggcaggaaactctctgactttggacaggaaacaagctatattgaagacaactgcaatcaaaatggtgccatatcactgatcttctcactcaaagaagaagttggtgcattggccaaagtattgcgcttatttgaggagaatgatgtaaacctgacccacattgaatctagaccttctcgtttaaagaaagatgagtatgaatttttcacccatttggataaacgtagcctgcctgctctgacaaacatcatcaagatcttgaggcatgacattggtgccactgtccatgagctttcacgagataagaagaaagacacagtgccctggttcccaagaaccattcaagagctggacagatttgccaatcagattctcagctatggagcggaactggatgctgaccaccctggttttaaagatcctgtgtaccgtgcaagacggaagcagtttgctgacattgcctacaactaccgccatgggcagcccatccctcgagtggaatacatggaggaagaaaagaaaacatggggcacagtgttcaagactctgaagtccttgtataaaacccatgcttgctatgagtacaatcacatttttccacttcttgaaaagtactgtggcttccatgaagataacattccccagctggaagacgtttctcaattcctgcagacttgcactggtttccgcctccgacctgtggctggcctgctttcctctcgggatttcttgggtggcctggccttccgagtcttccactgcacacagtacatcagacatggatccaagcccatgtatacccccgaacctgacatctgccatgagctgttgggacatgtgcccttgttttcagatcgcagctttgcccagttttcccaggaaattggccttgcctctctgggtgcacctgatgaatacattgaaaagctcgccacaatttactggtttactgtggagtttgggctctgcaaacaaggagactccataaaggcatatggtgctgggctcctgtcatcctttggtgaattacagtactgcttatcagagaagccaaagcttctccccctggagctggagaagacagccatccaaaattacactgtcacggagttccagcccctgtattacgtggcagagagttttaatgatgccaaggagaaagtaaggaactttgctgccacaatacctcggcccttctcagttcgctacgacccatacacccaaaggattgaggtcttggacaatacccagcagcttaagattttggctgattccattaacagtgaaattggaatcctttgcagtgccctccagaaaataaagtag

SEQ ID NO: 12 - DNA, 1359 bp, Artificial Sequence
  Synthetic Oligonucleotide
atgagcactgctgtgctggagaaccctggcctgggcaggaaactgagtgactttggccaggagaccagctacattgaggacaactgcaaccagaatggagccatcagcctgatcttcagcctgaaggaggaggtgggagccctggccaaggtgctgaggctgtttgaggagaatgatgtgaacctgacccacattgagagcaggcccagcaggctgaagaaggatgagtatgagttcttcacccacctggacaagaggagcctgcctgccctgaccaacatcatcaagatcctgaggcatgacattggagccacagtgcatgagctgagcagggacaagaagaaggacacagtgccctggttccccaggaccatccaggagctggacaggtttgccaaccagatcctgagctatggagctgagctggatgctgaccaccctggcttcaaggaccctgtgtacagggccaggaggaagcagtttgctgacattgcctacaactacaggcatggccagcccatccccagggtggagtacatggaggaggagaagaagacctggggcacagtgttcaagaccctgaagagcctgtacaagacccatgcctgctatgagtacaaccacatcttccccctgctggagaagtactgtggcttccatgaggacaacatcccccagctggaggatgtgagccagttcctgcagacctgcacaggcttcaggctgaggcctgtggctggcctgctgagcagcagggacttcctgggaggcctggccttcagggtgttccactgcacccagtacatcaggcatggcagcaagcccatgtacacccctgagcctgacatctgccatgagctgctgggccatgtgcccctgttcagtgacaggagctttgcccagttcagccaggagattggcctggccagcctgggagcccctgatgagtacattgagaagctggccaccatctactggttcacagtggagtttggcctgtgcaagcagggagacagcatcaaggcctatggagctggcctgctgagcagctttggagagctgcaatactgcctgagtgagaagcccaagctgctgcccctggagctggagaagacagccatccagaactacacagtgacagagttccagcccctgtactatgtggctgagagcttcaatgatgccaaggagaaagtgaggaactttgctgccaccatccccaggcccttcagtgtgaggtatgacccctacacccagaggattgaggtgctggacaacacccagcagctgaagatcctggctgacagcatcaacagtgagattggcatcctgtgcagtgccctgcagaagatcaagtag

SEQ ID NO: 13 - DNA, 1359 bp, Artificial Sequence
  Synthetic Oligonucleotide
atgtccactgcagtcctggagaacccaggcttgggcaggaaactctctgactttggacaggagaccagctatattgaagacaactgcaaccaaaatggtgccatctccctgatcttctccctcaaagaggaagtgggtgcattggccaaagtcttgaggctttttgaggagaatgatgtcaacctgacccacattgagtctagaccttctaggcttaagaaagatgagtatgagtttttcacccacttggataaaaggagcctgcctgctctgaccaacatcatcaagatcttgaggcatgacattggtgccactgtccatgagctttccagggataagaagaaagacacagtgccctggttcccaagaaccatccaagagctggacagatttgccaaccagatcctcagctatggagcagaactggatgctgaccaccctggcttcaaagatcctgtgtacagggcaagaagaaagcagtttgctgacattgcctacaactacaggcatgggcagcccatccctagggtggaatacatggaggaggaaaagaaaacctggggcacagtgttcaagaccctgaagtccttgtataaaacccatgcttgctatgagtacaaccacatctttccacttcttgagaagtactgtggcttccatgaagataacatcccccagctggaggatgtgtctcagttcctgcagacctgcactggcttcaggctcaggcctgtggctggcctgctttcctctagagatttcttgggtggcctggccttcagggtcttccactgcacacagtacatcagacatggatccaagcccatgtatacccctgaacctgacatctgccatgagctgttgggacatgtgcccttgttttcagataggagctttgcccagttctcccaggagattggccttgcctctctgggtgcacctgatgaatacattgagaagctggccaccatctactggttcactgtggagtttgggctctgcaaacaaggagactccatcaaggcatatggtgctgggctcctgtcatcctttggtgaacttcagtactgcctttcagagaagccaaagcttctccccctggagctggagaagacagccatccaaaactacactgtcacagagttccagcccctctattatgtggcagagagcttcaatgatgccaaggagaaagtcaggaactttgctgccaccatacctagacccttctcagtgaggtatgacccatacacccaaaggattgaggtcttggacaacacccagcaacttaagatcttggctgattccatcaacagtgagattggaatcctttgcagtgccctccagaaaatcaagtag

SEQ ID NO: 14 - DNA, 1359 bp, Artificial Sequence
  Synthetic Oligonucleotide
atgtccactgctgtcctggagaacccaggcttgggcaggaagctctctgactttgggcaggagaccagctacattgaggacaactgcaaccagaatggggccatctccctgatcttctccctcaaggaggaagtgggggccctggccaaggtgttgaggctgtttgaggaaaatgatgtgaacctgacccacattgagtccagaccctccaggctgaagaaggatgagtatgagttcttcacccacttggacaagaggagcctgcctgccctgaccaacatcatcaaaatcttgaggcatgacattggggccactgtccatgagctgtccagggacaagaaaaaggacacagtgccctggttccccagaaccatccaggagctggacagatttgccaaccagatcctcagctatggggctgagctggatgctgaccaccctggcttcaaggaccctgtgtacagggccagaagaaagcagtttgctgacattgcctacaactacaggcatgggcagcccatccccagggtggagtacatggaggaagagaaaaagacctggggcacagtgttcaagaccctgaagtccttatacaagacccatgcctgctatgagtacaaccacatcttccccctcctggagaagtactgtggcttccatgaggacaacatcccccagctggaggatgtctcccagttcctgcagacctgcactggcttcaggctcaggcctgtggctggcctcctgtcctccagagacttcttgggaggcctggccttcagggtcttccactgcacacagtacatcagacatggctccaagcccatgtacacccctgagcctgacatctgccatgagctgttgggccatgtgcccttgttctcagacaggagctttgcccagttctcccaggagattggcctggcctccctgggagcccctgatgagtacattgagaagctggccaccatctactggttcactgtggagtttgggctctgcaagcagggggactccatcaaggcctatggggctgggctcctgtcatcctttggggagctgcaatactgcctgtcagagaagcccaagctgctccccctggagctggagaagacagccatccagaactacactgtcactgagttccagcccctctactatgtggctgagagtttcaatgatgccaaggagaaagtgaggaactttgctgccaccatccctagacccttctcagtcaggtatgacccctacacccagaggattgaggtcttggacaacacccagcagctgaagatcttggctgactccatcaacagtgagattggcatcctgtgcagtgccctccagaagatcaagtag

SEQ ID NO: 15 - DNA, 1359 bp, Artificial Sequence
  Synthetic Oligonucleotide
atgagcaccgccgtgctggagaaccccggcctgggccgcaagctgagcgacttcggccaggagaccagctacatcgaggacaactgcaaccagaacggcgccatcagcctgatcttcagcctgaaggaggaggtgggcgccctggccaaggtgctgcgcctgttcgaggagaacgacgtgaacctgacccacatcgagagccgccccagccgcctgaagaaggacgagtacgagttcttcacccacctggacaagcgcagcctgcccgccctgaccaacatcatcaagatcctgcgccacgacatcggcgccaccgtgcacgagctgagccgcgacaagaagaaggacaccgtgccctggttcccccgcaccatccaggagctggaccgcttcgccaaccagatcctgagctacggcgccgagctggacgccgaccaccccggcttcaaggaccccgtgtaccgcgcccgccgcaagcagttcgccgacatcgcctacaactaccgccacggccagcccatcccccgcgtggagtacatggaggaggagaagaagacctggggcaccgtgttcaagaccctgaagagcctgtacaagacccacgcctgctacgagtacaaccacatcttccccctgctggagaagtactgcggcttccacgaggacaacatcccccagctggaggacgtgagccagttcctgcagacctgcaccggcttccgcctgcgccccgtggccggcctgctgagcagccgcgacttcctgggcggcctggccttccgcgtgttccactgcacccagtacatccgccacggcagcaagcccatgtacacccccgagcccgacatctgccacgagctgctgggccacgtgcccctgttcagcgaccgcagcttcgcccagttcagccaggagatcggcctggccagcctgggcgcccccgacgagtacatcgagaagctggccaccatctactggttcaccgtggagttcggcctgtgcaagcagggcgacagcatcaaggcctacggcgccggcctgctgagcagcttcggcgagctgcagtactgcctgagcgagaagcccaagctgctgcccctggagctggagaagaccgccatccagaactacaccgtgaccgagttccagcccctgtactacgtggccgagagcttcaacgacgccaaggagaaggtgcgcaacttcgccgccaccatcccccgccccttcagcgtgcgctacgacccctacacccagcgcatcgaggtgctggacaacacccagcagctgaagatcctggccgacagcatcaacagcgagatcggcatcctgtgcagcgccctgcagaagatcaagtag

SEQ ID NO: 16 - DNA, 1362 bp, Artificial Sequence
  Synthetic Oligonucleotide
atggcagctgttgtcctggagaacggagtcctgagcagaaaactctcagactttgggcaggaaacaagttacatcgaagacaactccaatcaaaatggtgctgtatctctgatattctcactcaaagaggaagttggtgccctggccaaggtcctgcgcttatttgaggagaatgagatcaacctgacacacattgaatccagaccttcccgtttaaacaaagatgagtatgagtttttcacctatctggataagcgtagcaagcccgtcctgggcagcatcatcaagagcctgaggaacgacattggtgccactgtccatgagctttcccgagacaaggaaaagaacacagtgccctggttcccaaggaccattcaggagctggacagattcgccaatcagattctcagctatggagccgaactggatgcagaccacccaggctttaaagatcctgtgtaccgggcgagacgaaagcagtttgctgacattgcctacaactaccgccatgggcagcccattcctcgggtggaatacacagaggaggagaggaagacctggggaacggtgttcaggactctgaaggccttgtataaaacacatgcctgctacgagcacaaccacatcttccctcttctggaaaagtactgcggtttccgtgaagacaacatcccgcagctggaagatgtttctcagtttctgcagacttgtactggtttccgcctccgtcctgttgctggcttactgtcgtctcgagatttcttgggtggcctggccttccgagtcttccactgcacacagtacattaggcatggatctaagcccatgtacacacctgaacctgatatctgtcatgaactcttgggacatgtgcccttgttttcagatagaagctttgcccagttttctcaggaaattgggcttgcatcgctgggggcacctgatgagtacattgagaaactggccacaatttactggtttactgtggagtttgggctttgcaaggaaggagattctataaaggcatatggtgctgggctcttgtcatcctttggagaattacagtactgtttatcagacaagccaaagctcctgcccctggagctagagaagacagcctgccaggagtatactgtcacagagttccagcctctgtactatgtggccgagagtttcaatgatgccaaggagaaagtgaggacttttgctgccacaatcccccggcccttctccgttcgctatgacccctacactcaaagggttgaggtcctggacaatactcagcagttgaagattttagctgactccattaatagtgaggttggaatcctttgccatgccctgcagaaaataaagtcatag

SEQ ID NO: 17 - DNA, 1359 bp, Artificial Sequence
  Synthetic Oligonucleotide
atgagcacagcagtcctggagaaccctgggcttgggaggaaactgagtgactttgggcaagagacctcctacattgaggataactgcaatcagaatggagccatcagcctcatcttctccctgaaagaggaggtgggggccctggccaaagtcctcagactctttgaggagaatgacgtgaacctgacccacattgagagcagacccagtaggctcaagaaggatgagtatgaattcttcacccacctggacaagaggagcctgcctgccctcaccaacatcatcaaaatcctgaggcatgacattggggccactgtccatgaactgtccagagacaagaagaaagacacagtcccctggtttccaaggaccatccaagagctggaccgctttgccaaccagatcctctcctatggggctgagctggatgctgaccaccctggcttcaaggacccagtgtaccgggccagaaggaagcagtttgctgatattgcctacaattacagacatggccagcccatccccagagtggagtacatggaggaagagaaaaagacctggggcacagtgttcaaaaccctgaagtccctctacaagacccatgcctgctatgagtacaaccacatctttcccctgctggagaagtactgtgggttccatgaggacaatatccctcagctggaagatgtgtcccagttcctgcagacctgcactggctttaggctgaggcctgtggctggcctgctcagctctagggacttcctgggggggctggccttcagagtcttccactgcacccagtacatccgccatgggagcaagcccatgtacaccccagagccagacatctgccatgagctgctgggccatgtgcccctcttctcagacagaagctttgcccagttcagccaagaaattggactggcctccctgggtgcccctgatgaatacatagaaaagctggctaccatctactggttcacagtggaatttggcctctgcaagcaaggggactccatcaaggcctatggagctgggctcctgagctcctttggagagctccaatactgcctgtctgagaagcccaagctcctgcccctggagcttgagaagacagccatccagaactacactgtgactgagttccagcctctgtactacgtggcagagtccttcaatgatgccaaggagaaggtgaggaactttgcagccaccattcctaggcccttctctgtgaggtatgacccctacacacagaggattgaagtgctggacaacacccagcagctcaagatcctggcagacagcatcaactcagagattggcatcctgtgctctgccctccagaagattaagtag

SEQ ID NO: 18 - DNA, 1359 bp, Artificial Sequence
  Synthetic Oligonucleotide
atgagcacagcagtgttggagaaccctgggcttgggaggaaactgtctgactttggacaagagacctcctacatagaagacaactgcaatcagaatggagccatctccctcatcttcagcctcaaggaagaggtgggggccctggccaaagtcctgaggctctttgaggagaatgacgtgaacctgacccacattgagagtaggccctcccggctgaagaaggatgaatatgaattcttcacccacctggataagaggagcctgcctgccctcaccaacatcatcaaaatcctgaggcatgacattggggccactgtgcatgagctctcaagggacaagaagaaagacacagtcccctggttccctaggaccatccaagagctggaccgctttgccaaccagatcctctcctatggggctgagctggatgctgaccaccctggcttcaaggacccagtgtaccgggccagaaggaagcagtttgcagatattgcctacaattacagacatggccagcccatcccaagggtggaatacatggaggaagaaaaaaagacctggggcactgtcttcaaaaccctgaagagcctgtacaagacccatgcctgctatgagtacaaccacatctttcccctgctggagaagtactgtgggttccatgaggacaatatccctcagctggaggatgtgtcccagttcctccagacctgcactggctttaggctgaggcctgtggctggcctgctgtccagcagagacttcctggggggcttggccttcagagtgttccactgcacccagtacatccgccatgggagcaagcccatgtacaccccagagccagacatctgccatgaactgctgggccatgtgcccctcttcagtgacagaagctttgcccagttctcccaagaaattggactggcctccctgggtgcccctgatgagtacattgagaagctggccaccatctactggtttacagtggagtttggcctctgcaagcaaggggactccatcaaggcctatggagctgggctgctcagctcctttggggagctgcaatactgcctgagtgagaaacccaagctcctgcccctggagctggaaaagacagccatccagaactacacagtcacagagttccagcctctctactatgtggcagagagcttcaatgatgccaaggagaaggtgaggaactttgctgccaccatccccagacccttctctgtgaggtatgacccctacactcagaggattgaagtgctggacaacacccagcagctcaagattctggctgatagcatcaactcagagattggcatcctgtgctctgccctgcagaagatcaagtag

SEQ ID NO: 19 - DNA, 1359 bp, Artificial Sequence
  Synthetic Oligonucleotide
atgagcacagcagtgttggagaaccctgggcttgggaggaaactgtctgactttgggcaagagacctcctacattgaggacaactgcaatcagaatggggccatcagcctcatcttttccctgaaagaggaggtgggggccctggccaaagtcctgaggctctttgaggagaatgacgtgaacctgacccacattgagagtaggccctctaggctgaagaaggatgaatatgaattcttcacccacctggacaagaggagcctgcctgccctcaccaacatcatcaaaatcctgaggcatgacattggagccactgtccatgagctgtccagagataagaagaaagacacagtcccctggtttccccgcaccatccaagagcttgaccgctttgccaaccagatcctgagctatggggcagagctggatgctgaccaccctggcttcaaggacccagtgtacagagccagaaggaagcagtttgctgatattgcctacaattaccgccatggacagcccatcccaagggtggaatacatggaggaagagaaaaagacctggggcacagtcttcaaaaccctgaagtccctctacaagacccatgcctgctatgagtacaaccacatcttccccctgctggagaagtactgtggcttccatgaagacaatatccctcagctggaggatgtgtcccagttcctgcagacctgcactgggttccgcctgagacctgtggctgggctcctctccagcagagacttcctggggggcttggccttcagagtgttccactgcacccagtacatcagacatggcagcaagcccatgtacaccccagagcctgacatctgccatgagctcctgggccatgtgcccctcttcagtgacagaagctttgcccagttctcccaagaaattgggctggcctccctgggtgcccctgatgagtacatagaaaagctggctaccatctactggttcacagtggagtttggcctgtgcaagcaaggggacagcatcaaggcctatggagctggcctgctcagctcctttggagagctccaatactgcctgagtgagaagcccaagctcctgcccctggaactggagaagacagccatccagaactacactgtgactgagttccagcctctgtactatgtggctgagagcttcaatgatgccaaggagaaggtgaggaactttgcagccaccatccccagacccttctctgtgaggtatgacccctacacacagaggattgaagtgctggacaacacccagcagctcaagatcctggcagactccatcaactcagagattggcatcctctgcagtgccctccagaagattaagtag

SEQ ID NO: 20 - DNA, 1359 bp, Artificial Sequence
  Synthetic Oligonucleotide
atgagcacagcagtcctggagaaccctgggcttgggaggaaactgtctgactttgggcaagagacctcctacatagaagacaactgcaatcagaatggagccatcagcctcatcttctccctgaaagaggaggtgggggccctggcaaaggtgctgaggctgtttgaggagaatgatgtgaacctgacccacattgaatctaggccctcccggctgaagaaggatgaatatgaattcttcacccacctggacaagaggagcctgcctgccctcaccaacatcatcaaaatcctgaggcatgacattggggccactgtccatgaactgtccagagataagaagaaagacacagtcccctggttccctaggaccatccaagagctggacagatttgccaaccagatcctctcctatggggctgagctggatgctgaccaccctggcttcaaggaccctgtctatagggccagaaggaagcagtttgctgatattgcctacaattacagacatggccagcccatcccaagggtggaatacatggaggaagagaaaaagacctggggcacagtcttcaaaaccctgaagtccctctacaagacccatgcctgctatgagtacaaccacatcttccccctgctggaaaagtactgtgggttccatgaggacaatatccctcagctggaggatgtctcccagttcctccagacctgcactggcttccgcctgagacctgtggctgggctcctgagcagcagagacttcctgggggggctggccttcagagtgttccactgcacacagtacatccgccatgggagcaagcccatgtacaccccagagccagacatctgccatgagctgctgggccatgtgcccctcttcagtgaccgcagctttgcccagttcagccaagaaattggactggcctccctgggtgcccctgatgagtacattgagaagctggccaccatctactggtttactgtggagtttggcctctgcaagcaaggggacagcatcaaggcctatggagctggcctgctcagctcctttggagagctccaatactgcctgagtgagaaacccaagctcctgcccctggagctggagaagacagccatccagaactacacagtgacagagttccagcctctgtactacgtggcagagagcttcaatgatgccaaggagaaggtcagaaactttgcagccaccatccccagacccttctctgtgaggtatgacccctacactcagaggattgaggtcttggacaacacccagcagctcaagattctggctgactccatcaactcagagattggcatcctgtgctctgccctgcagaagatcaagtaa

SEQ ID NO: 21 - DNA, 1359 bp, Artificial Sequence
  Synthetic Oligonucleotide
atgagcacagcagtcctggagaaccctgggcttgggaggaagctctctgactttgggcaagagacctcctacatagaagacaactgcaatcagaatggagccatcagcctcatcttctccctcaaggaagaagtgggggccctggcaaaggtcctgaggctctttgaggagaatgatgtcaacctgacccacattgagtctaggcccagcagactgaagaaggatgaatatgaattcttcacccacctggacaagaggagcctgcctgccctcaccaacatcatcaaaatcctgaggcatgacattggggccactgtccatgagctctccagagacaaaaagaaggacaccgtcccctggttccctaggaccatccaagaactggaccgctttgccaaccagatcctctcctatggggcagagctggatgctgaccaccctggcttcaaagacccagtgtacagagccagaaggaagcagtttgcagatattgcctacaattaccgccatggacagcccatcccaagggtggaatacatggaagaggagaagaaaacctggggcacagtgttcaagaccctgaagagcctgtacaagacccatgcctgctatgagtacaaccacatctttcccctgctggaaaagtactgtgggttccatgaggacaatatccctcagctggaggatgtgtcccagttcctccagacctgcactggctttaggctgaggcctgtggctggcctgctgtccagtagggacttcctggggggcttggccttcagagtcttccactgcacccagtacatcagacatggcagcaagcccatgtacaccccagagcctgacatctgccatgaactcctgggccatgtgcccctcttcagtgacagatcctttgcccagttcagccaagagatcgggctggcctccctgggtgcccctgatgagtacattgaaaaactggccaccatctactggtttactgtggagtttggcctctgcaagcaaggggacagcatcaaggcctatggagctgggctcctcagcagctttggagagctccaatactgcctgtctgagaaacccaagctgctccccctggagctggagaagacagccatccagaactacacagtcacagagttccagcctctctactacgtggctgagagcttcaatgatgccaaggagaaggtgaggaactttgctgccaccatccccagacccttctctgtgaggtatgacccctacactcagaggattgaggtgctggacaacacccagcagctcaagattctggctgactccatcaactcagagattggcatcctgtgctctgccctgcagaagatcaagtaa

SEQ ID NO: 22 - DNA, 1359 bp, Artificial Sequence
  Synthetic Oligonucleotide
atgagcacagcagtgttggagaaccctgggcttgggaggaaactgtctgactttgggcaagagacctcctacattgaagacaactgcaatcagaatggagccatcagcctcatcttttccctgaaagaggaggtgggggccctggccaaagtcctgaggctctttgaggagaatgacgtgaacctgacccacattgagagtaggcccagcagactgaagaaggatgaatatgaattcttcacccacctggacaagaggagcctgcctgccctcaccaacatcatcaaaatcctgaggcatgacattggggccactgtccacgagctctcaagggacaagaagaaagacacagtcccctggtttccaaggaccatccaagagcttgaccgctttgccaaccagatcctctcctatggggctgagctggatgctgaccaccctggctttaaggacccagtgtatagggccagaaggaagcagtttgctgatattgcctacaactacagacatggccagcccatcccaagggtggaatacatggaggaagagaaaaagacctggggcacagtgttcaagaccctgaagagcctgtacaagacccatgcctgctatgagtacaaccacatcttccccctgctggaaaagtactgtggcttccatgaggacaatatccctcagctggaggatgtgtcccagttcctccagacctgcactgggtttaggctgaggcctgtggctgggctcctcagcagccgggacttcctgggggggctggccttcagagtcttccactgcacccagtacatccgccatgggagcaagcccatgtacaccccagagccagacatctgccatgaactgctgggccatgtgcccctcttcagtgacagatcctttgcccagttctcccaagaaattggactggcctccctgggtgcccctgatgagtacatagagaagctggctaccatctactggttcacagtggagtttggcctctgcaagcaaggggactccatcaaggcctatggagctggcctgctgtccagctttggagagctgcagtattgcctgagtgagaaacccaagctcctgcccctggagctggagaagacagccatccagaactatactgtgactgagttccagcctctctactatgtggcagagagcttcaatgatgccaaggagaaggtgaggaactttgcagccaccatccccagacccttctctgtgaggtatgacccctacactcagaggatagaagtgctggacaacacccagcagctcaagatcctggcagacagcatcaactcagagattggcatcctgtgctctgccctccagaagattaagtaa

SEQ ID NO: 23 - DNA, 1359 bp, Artificial Sequence
  Synthetic Oligonucleotide
atgtccactgctgttctggagaaccctggactggggaggaagctctctgactttgggcaagagacctcctacattgaggacaactgcaaccagaatggggccatcagcctcatcttctccctgaaagaggaggtgggggccctggccaaggtcctgaggctctttgaggagaatgatgtgaacctgactcacattgagagccggcccagtaggctgaagaaggatgagtatgaattcttcacccacctggataagaggagcctgcctgccctgaccaacatcatcaaaatcctgaggcatgacattggagccactgtccacgagctctcaagggacaagaagaaagacactgtgccctggtttccaaggaccatccaagagctggacagatttgccaatcagatcctctcctatggggcagagctggatgctgaccaccctggcttcaaggaccctgtctatagggctaggaggaagcagtttgcagatattgcctacaattaccgccatggacagcccatcccaagggtggagtacatggaggaagagaaaaagacctggggcacagtcttcaaaaccctgaagtccctctacaagacccacgcctgctatgagtacaaccacatcttccccctgctggagaagtactgtgggttccatgaagacaatatcccccagcttgaggatgtctcccagttcctccagacctgtactggctttaggctgaggcctgtggctgggctcctgtccagcagagacttcctggggggcttggccttcagagtgttccactgcacacagtacatcagacatggcagcaagcccatgtacaccccagagccagacatctgccatgagctgctgggccatgtccccctcttcagtgaccgcagctttgcccagttcagccaagaaattgggctggcctccctgggggctcctgatgaatacatagagaagctggccaccatctactggttcacagttgagtttggcctctgcaagcaaggggacagcatcaaggcctatggagctggcctgctcagctcctttggagagctgcagtattgtctgtctgagaagcccaagctcctgcccctggagctggaaaagacagccatccagaactacacagtgacagagttccagcctctgtactatgtggctgagtccttcaatgatgccaaggagaaggtgaggaattttgctgccaccattcccagacccttctctgtgaggtatgacccctacactcagaggattgaagtgctggacaacacccagcagctcaagatcctggcagactccatcaactcagagattggcatcctgtgttctgccctccagaagatcaagtga

SEQ ID NO: 24 - DNA, 1359 bp, Artificial Sequence
  Synthetic Oligonucleotide
atgagcacagcagttctggagaaccctggactggggaggaagctgtctgactttggacaagagacctcctacatagaggacaactgcaatcagaatggagccatcagcctcatcttcagcctcaaggaggaagtgggggccctggccaaggtcctgaggctgtttgaggagaatgatgtgaacctgactcacattgagagtaggccctcaaggctcaagaaggatgagtatgagttcttcacccacctggataagaggtccctgcctgccctgaccaacatcatcaaaatcctgcgccatgacattggggccacagtgcacgagctctcaagggacaagaagaaagacacagtcccctggtttccccgcaccatccaagagctggacagatttgccaaccagatcctctcctatggggctgagctggatgctgaccaccctggctttaaggacccagtgtatagggccagaaggaagcagtttgctgatattgcctacaattacagacatggccagcccatccccagagttgagtacatggaggaagagaaaaagacctggggcactgttttcaagaccctgaagtccctctacaagacccacgcctgctatgagtacaaccacatcttcccactgctggaaaagtactgtggcttccatgaggacaatatccctcagctggaggatgtctcccagttcctccagacctgtactgggtttaggctgaggcctgtagctggcctgctcagctctagggacttcctgggagggctggccttccgggtcttccactgcacccagtacatcagacatgggagcaagcccatgtacaccccagagccagacatctgccatgagctgctgggccatgtgcccctcttctcagataggtcctttgcccagttctcccaagagataggcctggcatccctgggtgcccctgatgagtacattgagaagctggccaccatctactggttcactgtggagtttggcctctgcaagcaaggggacagcatcaaggcctatggagctgggctcctgtccagctttggggagctgcagtattgtctgagtgagaagcccaagctcctgccactggagctggagaagacagccatccagaactacacagtcacagagttccagcctctgtactatgtggcagagagcttcaatgatgccaaggagaaggtgaggaattttgcagccaccatcccaagacccttctctgtgaggtatgacccctacactcagaggattgaggtgctggacaacacccagcagctcaagattctggctgactccatcaactcagagattggcatcctgtgttctgccctccagaagatcaagtga

SEQ ID NO: 25 - DNA, 1359 bp, Artificial Sequence
  Synthetic Oligonucleotide
atgagcacagctgtgctggagaaccctgggcttggaaggaagctcagtgactttggccaagagacctcctacattgaggacaactgcaatcagaatggagccatcagcctcatcttttccttgaaggaagaagtgggggccttggccaaagtcctgaggctgtttgaggagaatgacgtcaacctgactcacattgaatctaggccttcaaggctcaagaaggatgagtatgaattcttcacccacctggacaagaggagcctgcctgctctgaccaacatcatcaaaatcttgaggcatgacattggagcaacagtccacgagcttagcagagacaaaaagaaagacaccgtgccctggttcccaagaaccattcaagagttggataggtttgccaaccagatcctctcctatggggctgagctggatgctgaccaccctggctttaaggaccctgtgtatagagccagaagaaagcagtttgctgatattgcctacaattacagacatggacagcccatccccagagtggagtacatggaggaagagaaaaaaacctggggcactgtcttcaagaccctgaaatctctgtacaagacacatgcctgctatgagtacaaccacatcttccctctgttggagaagtactgtggcttccatgaagataacattccccagcttgaggatgtgtctcaatttctccagacctgcactggattcagactcagaccagtggctggcctgctgtccagtagggacttcctgggaggactggcctttagggtgttccactgcacacagtacatcagacacggcagcaagcccatgtacacaccagagccagacatctgccatgagctcctgggccatgtccccctcttctctgacagatcctttgcccagttctcccaagaaattggtctggcttccctgggtgcccctgatgaatatatagaaaagctggccaccatctactggtttacagtggaatttgggctctgcaaacaaggagactccattaaggcctatggagctgggctgctcagcagctttggagagctgcaatactgcctgtctgaaaaacccaagcttctgcccctggaactggagaaaacagcaatccagaactacactgtgactgagttccagcctctctactacgtggcagagagcttcaatgatgccaaggagaaggtgagaaactttgcagccactatcccaaggcccttcagtgttagatatgacccctacacccagaggattgaggtgcttgacaatactcagcagctgaagattctggcagattccatcaactcagagattggcatcctgtgttctgccctgcagaagatcaagtaa

SEQ ID NO: 26 - skipped

SEQ ID NO: 27 - DNA, 1359 bp, Artificial Sequence
  Synthetic Oligonucleotide
atgtccactgctgtgttggagaaccctggacttggcagaaaactgagtgactttgggcaagagacctcctacattgaagataactgcaatcagaatggagccatttccctcatcttctccctgaaggaagaagtgggggccctggccaaagtcctgcgcctgtttgaggagaatgacgtcaacctgacccacatcgaatctaggccttcaaggttgaagaaggatgaatatgagttcttcacacacctggataagaggagcctgcctgccctcaccaacatcatcaaaatcttgaggcatgacattggagcaacagtccacgagctgagcagagacaagaagaaagacaccgtcccctggtttcccagaaccatccaagaacttgaccgcttcgccaaccagatcctgtcctatggcgcagagcttgatgctgaccaccctgggttcaaggacccagtgtacagagccagaaggaagcagtttgcagatattgcctacaattaccgccatggccagcccatcccaagagtggagtacatggaggaagagaaaaagacctggggcactgtgttcaaaaccctgaaaagcctctacaagactcacgcctgctatgaatacaaccacattttcccactgcttgagaagtactgtggcttccatgaggacaatatcccccagctggaggatgtgtctcaatttctgcagacctgcactggctttcggctgagacctgtggccggcctcctcagcagccgggacttcctgggaggcttggccttcagagtcttccactgcacacagtatatcagacatggaagcaagcccatgtacactccagagccagacatctgccatgaactgctgggccatgtgcccctcttctctgaccggagctttgcccagttcagccaagagattggccttgcctctctgggggctcctgatgagtacatcgagaagctggctaccatctactggttcaccgtggaatttggcctgtgcaaacaaggagactccatcaaggcctatggagctgggctgctctcctcctttggagagctccagtactgcctgtctgaaaaacccaagctcctgcccctggagctggaaaagacagccatccagaactacacagtgacagaattccagcctctgtactacgtggctgagagcttcaatgatgccaaggagaaggtgagaaactttgctgccaccattcctcggcccttttctgtgcgctatgacccctacacccaaagaattgaggtgctggacaacacccagcagctcaagattctggcagacagcatcaactcagagatcggcatcctctgctccgcccttcagaagatcaagtaa

(19) United States
(12) Patent Application Publication
(10) Pub. No.: US 2023/0055020 A1
(43) Pub. Date: Feb. 23, 2023

(54) ADENO ASSOCIATED VIRUS BASED GENE THERAPY FOR PHENYLKETONURIA

(71) Applicant: TAKEDA PHARMACEUTICAL COMPANY LIMITED, Osaka (JP)

(72) Inventors: Matthias KLUGMANN, Lexington, MA (US); Hanspeter ROTTENSTEINER, Lexington, MA (US); Franziska HORLING, Lexington, MA (US); Johannes LANGLER, Lexington, MA (US)

(21) Appl. No.: 17/792,100

(22) PCT Filed: Jan. 15, 2021
(86) PCT No.: PCT/IB2021/000008

§ 371 Date: Jul. 11, 2022

Related U.S. Application Data
(60) Provisional application No. 62/962,011, filed on Jan. 16, 2020.

(51) Int. Cl. C12N 15/86; A61K 48/00; C12N 9/02

(52) U.S. Cl. CPC C12N 15/86; A61K 48/005; C12N 9/0071; C12Y 114/16001; C12N 2750/14143; C12N 2800/22

(57) ABSTRACT

The present disclosure provides, among other things, a recombinant adeno-associated virus (rAAV) vector comprising an AAV8 capsid and a codon-optimized sequence encoding a human phenylalanine hydroxylase (PAH) enzyme. The disclosure also provides a method of treating a subject having phenylketonuria (PKU), comprising administering to the subject in need thereof a recombinant adeno-associated vims (rAAV) vector comprising an AAV8 capsid, and a promoter operably linked to a nucleic acid sequence that encodes PAH, and wherein administering results in a decrease in phenylalanine level in the subject.

### CROSS-REFERENCE TO RELATED APPLICATIONS

This application claims benefit of, and priority to, U.S. Ser. No. 62/962,011 filed on Jan. 16, 2020, the contents of which are incorporated herein.

### BACKGROUND

Phenylketonuria (PKU) is an autosomal recessive metabolic genetic disorder characterized by a mutation in the gene for the hepatic enzyme phenylalanine hydroxylase (PAH), rendering it nonfunctional. PAH is necessary to metabolize the amino acid phenylalanine (Phe) to the amino acid tyrosine. When PAH activity is reduced, phenylalanine accumulates and is converted into phenylpyruvate (also known as phenylketone). Left untreated, PKU can result in mental retardation, seizures and other serious medical problems. Currently, there is no cure for the disease and standard of care is through management of diet, minimizing foods that contain high amounts of protein.

The use of vectors that produce therapeutic proteins in vivo is desirable for the treatment of disease, but is limited by various factors including poor production of desired therapeutic proteins in vivo.

### SUMMARY OF THE INVENTION

The present invention provides, among other things, methods and compositions for the effective treatment of PKU using gene therapy. The present invention is based, in part, on the surprising discovery of successful treatment of PKU in an animal model of the disease using recombinant adeno-associated virus (rAAV) vectors comprising a codon-optimized human PAH. For example, as described in more detail in the examples section below, administration of rAAV vectors that encode PAH resulted in efficient protein expression. Furthermore, rAAV vectors encoding an AAV8 capsid and codon-optimized human PAH were particularly effective in decreasing the phenylalanine level and increasing the tyrosine and tryptophan levels in both plasma and brains of PKU mice. Thus, the present inventors have demonstrated that the gene therapy approach described herein can be highly effective in treating PKU.

In one aspect, the present invention provides a rAAV comprising a codon-optimized sequence encoding a human PAH, wherein the codon-optimized sequence has at least 70% identity to one of SEQ ID Nos: 11-27.

In some embodiments, the codon-optimized sequence has at least 75% identity to one of SEQ ID Nos: 11-27. In some embodiments, the codon-optimized sequence has at least 80% identity to one of SEQ ID Nos: 11-27. In some embodiments, the codon-optimized sequence has at least 85% identity to one of SEQ ID Nos: 11-27. In some embodiments, the codon-optimized sequence has at least 90% identity to one of SEQ ID Nos: 11-27. In some embodiments, the codon-optimized sequence has at least 95% identity to one of SEQ ID Nos: 11-27. In some embodiments, the codon-optimized sequence has at least 99% identity to one of SEQ ID Nos: 11-27.

In some embodiments, the codon-optimized sequence is identical to one of SEQ ID Nos: 11-27.

In some embodiments, the rAAV encodes an AAV8 capsid.

In some embodiments, the rAAV8 capsid is a modified AAV8 capsid with improved liver tropism compared to the wild-type AAV8 capsid.

In some embodiments, the AAV8 capsid has at least 70%, 75%, 80%, 85%, 90%, 95% or 99% identity to the wild-type AAV capsid.

In some embodiments, the rAAV further comprises a Woodchuck Posttranscriptional Regulatory Element (WPRE) sequence.

In some embodiments, the WPRE sequence is a naturally-occurring WPRE sequence.

In some embodiments, the WPRE sequence is a modified WPRE sequence. In some embodiments, the WPRE sequence is selected from a wild-type WPRE, WPRE3, or WPREmut6delATG.

In some embodiments, the rAAV further comprises a liver-specific promoter.

In some embodiments, the liver-specific promoter is a transthyretin promotor (TTR).

In some embodiments, the rAAV comprises a cis-acting regulatory module (CRM).

In some embodiments, the vector comprises one, two, three, four, five or more CRM repeats.

In some embodiments, the CRM is CRM8.

In some embodiments, the rAAV further comprises an intron upstream of the PAH sequence.

In some embodiments, the intron is a minute virus of mice (MVM) intron.

In one aspect, the present invention provides a method of treating PKU, comprising administering to a subject in need of treatment a rAAV comprising a codon-optimized sequence encoding a human phenylalanine hydroxylase (PAH), wherein the codon-optimized sequence has at least 70% identity to one of SEQ ID Nos: 11-27. In some embodiments, the codon-optimized sequence encoding PAH comprises a GC content of 40% and 80%. For example, in some embodiments, the codon-optimized sequence encoding PAH comprises a GC content of about 40%, 45%, 50%, 55%, 60%, 65%, 70%, 75%, or 80%. In some embodiments, the codon-optimized sequence encoding PAH comprises 10 or less CpG island sequences. For example, in some embodiments, the codon-optimized sequence encoding PAH comprises 10, 9, 8, 7, 6, 5, 4, 3, 2, 1 or 0 CpG island sequences. In some embodiments, the codon-optimized sequence encoding PAH comprises less than 6 CpG island sequences.

In some embodiments, administering the rAAV results in a decrease in plasma phenylalanine (Phe) level in the subject compared to a control.

In some embodiments, administering the rAAV results in an increase in plasma tyrosine level in the subject compared to a control.

In some embodiments, administering the rAAV results in an increase in plasma tryptophan level in the subject compared to a control.

In some embodiments, the control is the pre-treatment level of plasma Phe, plasma tyrosine, and/or plasma tryptophan in the subject.

In some embodiments, the control is a reference level of plasma Phe, plasma tyrosine, and/or plasma tryptophan based on historical data. For example, the historical data (e.g., tissue sample measurements, protein or mRNA measurements) can be obtained from other patients, the same patients (e.g., pre-treatment), or from healthy individuals.

In some embodiments, the rAAV is administered at dose of about 1×10¹⁰ vg/kg, about 1×10¹¹ vg/kg, about 1×10¹² vg/kg, about 1×10¹³ vg/kg, about 1×10¹⁴ vg/kg, or about 1×10¹⁵ vg/kg.

In some embodiments, the rAAV vectors are administered systemically.

In some embodiments, the rAAV vectors are administered intravenously.

### BRIEF DESCRIPTION OF THE DRAWINGS

FIGS. 1A-1D are a series of schematic representations of exemplary expression constructs comprising wild-type (wt) and codon-optimized (co) human PAH (hPAH) expressing sequences. The corresponding co hPAH sequences shown in the expression constructs are found in Table 2. ITR: inverted terminal repeat; hTTR: human transthyretin promoter; CRM: cis-acting regulatory module; MVM intron: minute virus of mice intron; BGH pA: Bovine growth hormone terminator+polyA; WPRE: woodchuck posttranscriptional regulatory element.

FIG. 2 shows a Western blot analysis of hPAH expression in HepG2 cells infected with rAAV8 vectors encoding wt or co hPAH. Protein bands representing hPAH protein are at ˜50 kD.

FIG. 3A is an exemplary graph that shows the plasma levels of Phe, Tyr and Trp at baseline, 1, 2, 3, 4 and 5 weeks post administration of rAAV. FIG. 3B depicts a series of bar graphs that show the in vivo transduction efficiency (hPAH DNA) and transcription efficiency (hPAH RNA) of the rAAV8 comprising either co hPAH or wt hPAH.

FIG. 4 shows an exemplary representation of coat color correction in PKU mice 3 weeks post treatment by gene therapy with rAAV8 encoding codon-optimized hPAH.

FIG. 5 shows a dose-dependent efficacy of rAAV8 vectors encoding codon-optimized hPAH in normalizing plasma levels of Phe, Tyr and Trp and in correcting coat color of PAH-KO mice at 0, 1, 2, 3, 4 and 5 weeks post treatment.

FIG. 6 is an exemplary graph which shows that the levels of Large Neutral Amino Acids (LNAAs) (phenylalanine, tyrosine, and tryptophan) and neurotransmitters (dopamine, serotonin and noradrenaline) are dysregulated in the brain of PAH-KO mice.

FIG. 7A is an exemplary graph that shows the levels of Phe, Tyr and Trp in brain tissue of PAH-KO mice at 5 weeks post treatment with rAAV8 vectors encoding co hPAH or in untreated PAH-KO mice. FIG. 7B is an exemplary graph that shows the levels of serotonin, noradrenaline and dopamine neurotransmitters in brain tissue of PAH-KO mice at 5 weeks post treatment with rAAV8 vectors encoding codon-optimized hPAH or in untreated PAH-KO mice.

FIG. 8 is an exemplary graph that shows plasma levels of Phe at baseline, 7, 14, 35, 56, 98, 140 and 182 days post administration of rAAV vectors encoding codon-optimized hPAH at various doses in PAH-KO mice. The Phe levels in mice treated with codon-optimized hPAH are compared to levels in C22-treated PAH-KO mice and C22-treated wt mice.

### DEFINITIONS

Adeno-associated virus (AAV): As used herein, the terms “adeno-associated virus” or “AAV” or recombinant AAV (“rAAV”) include, but are not limited to, AAV type 1, AAV type 2, AAV type 3 (including types 3A and 3B), AAV type 4, AAV type 5, AAV type 6, AAV type 7, AAV type 8, AAV type 9, AAV type 10, AAV type 11, avian AAV, bovine AAV, canine AAV, equine AAV, and ovine AAV (see, e.g., Fields et al., Virology, volume 2, chapter 69 (4th ed., Lippincott-Raven Publishers); Gao et al., J. Virology 78:6381-6388 (2004); Mori et al., Virology 330:375-383 (2004)). Typically, AAV can infect both dividing and non-dividing cells and can be present in an extrachromosomal state without integrating into the genome of a host cell. AAV vectors are commonly used in gene therapy. AAV also includes codon-optimized AAV.

Administering: As used herein, the terms “administering,” “delivering” or “introducing” are used interchangeably in the context of delivering rAAV vectors encoding PAH into a subject, by a method or route which results in efficient delivery of the rAAV vector. Various methods are known in the art for administering rAAV vectors, including for example intravenously, subcutaneously or transdermally. Transdermal administration of rAAV vector can be performed by use of a “gene gun” or biolistic particle delivery system. In some embodiments, the rAAV vectors are administered via non-viral lipid nanoparticles.

Animal: As used herein, the term “animal” refers to any member of the animal kingdom. In some embodiments, “animal” refers to humans, at any stage of development. In some embodiments, “animal” refers to non-human animals, at any stage of development. In certain embodiments, the non-human animal is a mammal (e.g., a rodent, a mouse, a rat, a rabbit, a monkey, a dog, a cat, a sheep, cattle, a primate, and/or a pig). In some embodiments, animals include, but are not limited to, mammals, birds, reptiles, amphibians, fish, insects, and/or worms. In some embodiments, an animal may be a transgenic animal, genetically-engineered animal, and/or a clone.

Approximately or about. As used herein, the term “approximately” or “about,” as applied to one or more values of interest, refers to a value that is similar to a stated reference value. In certain embodiments, the term “approximately” or “about” refers to a range of values that fall within 25%, 20%, 19%, 18%, 17%, 16%, 15%, 14%, 13%, 12%, 11%, 10%, 9%, 8%, 7%, 6%, 5%, 4%, 3%, 2%, 1%, or less in either direction (greater than or less than) of the stated reference value unless otherwise stated or otherwise evident from the context (except where such number would exceed 100% of a possible value).

Active: As used herein, the phrase “active” refers to a characteristic of any agent that has activity in a biological system, and particularly in an organism. For instance, an agent that, when administered to an organism, has a biological effect on that organism, is considered to be active or biologically active. In particular embodiments, where a peptide is active or biologically active, a portion of that peptide that shares at least one biological activity of the peptide is typically referred to as an “active” portion.

Functional equivalent or derivative: As used herein, the term “functional equivalent” or “functional derivative” denotes, in the context of a functional derivative of an amino acid sequence, a molecule that retains a biological activity (either functional or structural) that is substantially similar to that of the original sequence. A functional derivative or equivalent may be a natural derivative or is prepared synthetically. Exemplary functional derivatives include amino acid sequences having substitutions, deletions, or additions of one or more amino acids, provided that the biological activity of the protein is conserved. The substituting amino acid desirably has chemico-physical properties which are similar to that of the substituted amino acid. Desirable similar chemico-physical properties include, similarities in charge, bulkiness, hydrophobicity, hydrophilicity, and the like.

In vitro: As used herein, the term “in vitro” refers to events that occur in an artificial environment, e.g., in a test tube or reaction vessel, in cell culture, etc., rather than within a multi-cellular organism.

In vivo: As used herein, the term “in vivo” refers to events that occur within a multi-cellular organism, such as a human and a non-human animal. In the context of cell-based systems, the term may be used to refer to events that occur within a living cell (as opposed to, for example, in vitro systems).

IRES: As used herein, the term “IRES” refers to any suitable internal ribosome entry site sequence.

Isolated: As used herein, the term “isolated” refers to a substance and/or entity that has been (1) separated from at least some of the components with which it was associated when initially produced (whether in nature and/or in an experimental setting), and/or (2) produced, prepared, and/or manufactured by the hand of man. Isolated substances and/or entities may be separated from at least about 10%, about 20%, about 30%, about 40%, about 50%, about 60%, about 70%, about 80%, about 90%, about 95%, about 98%, about 99%, substantially 100%, or 100% of the other components with which they were initially associated. In some embodiments, isolated agents are more than about 80%, about 85%, about 90%, about 91%, about 92%, about 93%, about 94%, about 95%, about 96%, about 97%, about 98%, about 99%, substantially 100%, or 100% pure. As used herein, a substance is “pure” if it is substantially free of other components. As used herein, the term “isolated cell” refers to a cell not contained in a multi-cellular organism.

Polypeptide: The term “polypeptide,” as used herein refers a sequential chain of amino acids linked together via peptide bonds. The term is used to refer to an amino acid chain of any length, but one of ordinary skill in the art will understand that the term is not limited to lengthy chains and can refer to a minimal chain comprising two amino acids linked together via a peptide bond. As is known to those skilled in the art, polypeptides may be processed and/or modified.

Protein: The term “protein” as used herein refers to one or more polypeptides that function as a discrete unit. If a single polypeptide is the discrete functioning unit and does not require permanent or temporary physical association with other polypeptides in order to form the discrete functioning unit, the terms “polypeptide” and “protein” may be used interchangeably. If the discrete functional unit is comprised of more than one polypeptide that physically associate with one another, the term “protein” refers to the multiple polypeptides that are physically coupled and function together as the discrete unit.

Regulatory element: As used herein, the term “regulatory element” refers to transcriptional control elements, in particular non-coding cis-acting transcription control elements, capable of regulating and/or controlling transcription of a gene. Regulatory elements comprise at least one transcription factor binding site, for example at least one binding site for a tissue specific transcription factor. In embodiments described herein, regulatory elements have at least one binding site for a liver-specific transcription factor. Typically, regulatory elements increase or enhance promoter-driven gene expression when compared to the transcription of the gene from the promoter alone, without the regulatory elements. Thus, regulatory elements particularly comprise enhancer sequences, although it is to be understood that the regulatory elements enhancing transcription are not limited to typical far upstream enhancer sequences, but may occur at any distance of the gene they regulate. As is understood in the art, sequences regulating transcription may be situated either upstream (e.g., in the promoter region) or downstream (e.g., in the 3′UTR) of the gene that is regulated in vivo, and may be located in the immediate vicinity of the gene or further away. Regulatory elements can comprise either naturally occurring sequences, combinations of (parts of) such regulatory elements or several copies of a regulatory element, e.g., non-naturally occurring sequences. Accordingly, regulatory elements include naturally occurring and optimized or engineered regulatory elements to achieve a desired expression level.

Subject: As used herein, the term “subject” refers to a human or any non-human animal (e.g., mouse, rat, rabbit, dog, cat, cattle, swine, sheep, horse or primate). A human includes pre- and post-natal forms. In many embodiments, a subject is a human being. A subject can be a patient, which refers to a human presenting to a medical provider for diagnosis or treatment of a disease. The term “subject” is used herein interchangeably with “individual” or “patient.” A subject can be afflicted with or is susceptible to a disease or disorder but may or may not display symptoms of the disease or disorder.

Substantially: As used herein, the term “substantially” refers to the qualitative condition of exhibiting total or near-total extent or degree of a characteristic or property of interest. One of ordinary skill in the biological arts will understand that biological and chemical phenomena rarely, if ever, go to completion and/or proceed to completeness or achieve or avoid an absolute result. The term “substantially” is therefore used herein to capture the potential lack of completeness inherent in many biological and chemical phenomena.

Substantial homology: The phrase “substantial homology” is used herein to refer to a comparison between amino acid or nucleic acid sequences. As will be appreciated by those of ordinary skill in the art, two sequences are generally considered to be “substantially homologous” if they contain homologous residues in corresponding positions. Homologous residues may be identical residues. Alternatively, homologous residues may be non-identical residues will appropriately similar structural and/or functional characteristics. For example, as is well known by those of ordinary skill in the art, certain amino acids are typically classified as “hydrophobic” or “hydrophilic” amino acids, and/or as having “polar” or “non-polar” side chains. Substitution of one amino acid for another of the same type may often be considered a “homologous” substitution.

As is well known in this art, amino acid or nucleic acid sequences may be compared using any of a variety of algorithms, including those available in commercial computer programs such as BLASTN for nucleotide sequences and BLASTP, gapped BLAST, and PSI-BLAST for amino acid sequences. Exemplary such programs are described in Altschul, et al., basic local alignment search tool, J. Mol. Biol., 215(3): 403-410, 1990; Altschul, et al., Methods in Enzymology; Altschul, et al., “Gapped BLAST and PSI-BLAST: a new generation of protein database search programs”, Nucleic Acids Res. 25:3389-3402, 1997; Baxevanis, et al., Bioinformatics: A Practical Guide to the Analysis of Genes and Proteins, Wiley, 1998; and Misener, et al., (eds.), Bioinformatics Methods and Protocols (Methods in Molecular Biology, Vol. 132), Humana Press, 1999. In addition to identifying homologous sequences, the programs mentioned above typically provide an indication of the degree of homology. In some embodiments, two sequences are considered to be substantially homologous if at least 50%, 55%, 60%, 65%, 70%, 75%, 80%, 85%, 90%, 91%, 92%, 93%, 94%, 95%, 96%, 97%, 98%, 99% or more of their corresponding residues are homologous over a relevant stretch of residues. In some embodiments, the relevant stretch is a complete sequence. In some embodiments, the relevant stretch is at least 10, 15, 20, 25, 30, 35, 40, 45, 50, 55, 60, 65, 70, 75, 80, 85, 90, 95, 100, 125, 150, 175, 200, 225, 250, 275, 300, 325, 350, 375, 400, 425, 450, 475, 500 or more residues.

Substantial identity: The phrase “substantial identity” is used herein to refer to a comparison between amino acid or nucleic acid sequences. As will be appreciated by those of ordinary skill in the art, two sequences are generally considered to be “substantially identical” if they contain identical residues in corresponding positions. As is well known in this art, amino acid or nucleic acid sequences may be compared using any of a variety of algorithms, including those available in commercial computer programs such as BLASTN for nucleotide sequences and BLASTP, gapped BLAST, and PSI-BLAST for amino acid sequences. Exemplary such programs are described in Altschul, et al., Basic local alignment search tool, J. Mol. Biol., 215(3): 403-410, 1990; Altschul, et al., Methods in Enzymology; Altschul et al., Nucleic Acids Res. 25:3389-3402, 1997; Baxevanis et al., Bioinformatics: A Practical Guide to the Analysis of Genes and Proteins, Wiley, 1998; and Misener, et al., (eds.), Bioinformatics Methods and Protocols (Methods in Molecular Biology, Vol. 132), Humana Press, 1999. In addition to identifying identical sequences, the programs mentioned above typically provide an indication of the degree of identity. In some embodiments, two sequences are considered to be substantially identical if at least 50%, 55%, 60%, 65%, 70%, 75%, 80%, 85%, 90%, 91%, 92%, 93%, 94%, 95%, 96%, 97%, 98%, 99% or more of their corresponding residues are identical over a relevant stretch of residues. In some embodiments, the relevant stretch is a complete sequence. In some embodiments, the relevant stretch is at least 10, 15, 20, 25, 30, 35, 40, 45, 50, 55, 60, 65, 70, 75, 80, 85, 90, 95, 100, 125, 150, 175, 200, 225, 250, 275, 300, 325, 350, 375, 400, 425, 450, 475, 500 or more residues.

Suffering from: An individual who is “suffering from” a disease, disorder, and/or condition has been diagnosed with or displays one or more symptoms of the disease, disorder, and/or condition.

Therapeutically effective amount: As used herein, the term “therapeutically effective amount” of a therapeutic agent means an amount that is sufficient, when administered to a subject suffering from or susceptible to a disease, disorder, and/or condition, to treat, diagnose, prevent, and/or delay the onset of the symptom(s) of the disease, disorder, and/or condition. It will be appreciated by those of ordinary skill in the art that a therapeutically effective amount is typically administered via a dosing regimen comprising at least one unit dose.

Treating: As used herein, the term “treat,” “treatment,” or “treating” refers to any method used to partially or completely alleviate, ameliorate, relieve, inhibit, prevent, delay onset of, reduce severity of and/or reduce incidence of one or more symptoms or features of a particular disease, disorder, and/or condition. Treatment may be administered to a subject who does not exhibit signs of a disease and/or exhibits only early signs of the disease for the purpose of decreasing the risk of developing pathology associated with the disease.

The recitation of numerical ranges by endpoints herein includes all numbers and fractions subsumed within that range (e.g. 1 to 5 includes 1, 1.5, 2, 2.75, 3, 3.9, 4 and 5). It is also to be understood that all numbers and fractions thereof are presumed to be modified by the term “about.”

Various aspects of the invention are described in detail in the following sections. The use of sections is not meant to limit the invention. Each section can apply to any aspect of the invention. In this application, the use of “or” means “and/or” unless stated otherwise. As used herein, the singular forms “a”, “an”, and “the” include both singular and plural referents unless the context clearly dictates otherwise.

### DETAILED DESCRIPTION

The present invention provides, among other things, methods and compositions for treating PKU using rAAV vectors that encode wild type or codon-optimized phenylalanine hydroxylase (PAH). In particular, the present invention provides a method of treating PKU by administering a rAAV comprising a wild type or codon-optimized sequence encoding a human PAH at an effective dose such that at least one symptom or feature of PKU is reduced in intensity, severity, or frequency. The gene therapy method described herein was particularly effective in normalizing the phenylalanine level.

### Phenylketonuria (PKU)

The present invention may be used to treat a subject who is suffering from or susceptible to PKU. PKU is an autosomal recessive metabolic genetic disorder characterized by a mutation in the gene for the hepatic enzyme PAH, rendering it nonfunctional. PAH is necessary to metabolize the amino acid phenylalanine (Phe) to the amino acid tyrosine. When PAH activity is reduced, phenylalanine accumulates and is converted into phenylpyruvate (also known as phenylketone) which can be detected in the urine.

Phenylalanine is a large, neutral amino acid (LNAA). LNAAs compete for transport across the blood—brain barrier (BBB) via the large neutral amino acid transporter (LNAAT). Excess Phe in the blood saturates the transporter and tends to decrease the levels of other LNAAs in the brain. Because several of these other amino acids are necessary for protein and neurotransmitter synthesis, Phe buildup hinders the development of the brain, and can cause mental retardation.

In addition to hindered brain development, the disease can present clinically with a variety of symptoms including seizures, albinism, hyperactivity, stunted growth, skin rashes (eczema), microcephaly, and/or a “musty” odor to an affected baby's sweat and urine, due to phenylacetate, one of the ketones produced. Untreated children are typically normal at birth, but have delayed mental and social skills, have a head size significantly below normal, and often demonstrate progressive impairment of cerebral function. As the child grows and develops, additional symptoms including hyperactivity, jerking movements of the arms or legs, EEG abnormalities, skin rashes, tremors, seizures, and severe learning disabilities tend to develop. PKU is commonly included in the routine newborn screening panel of most countries that is typically performed 2-7 days after birth.

If PKU is diagnosed early enough, an affected newborn can grow up with relatively normal brain development, but only by managing and controlling Phe levels through diet, or a combination of diet and medication. All PKU patients must adhere to a special diet low in Phe for optimal brain development. The diet requires severely restricting or eliminating foods high in Phe, such as meat, chicken, fish, eggs, nuts, cheese, legumes, milk and other dairy products. Starchy foods, such as potatoes, bread, pasta, and corn, must be monitored. Infants may still be breastfed to receive all of the benefits of breastmilk, but the quantity must also be monitored and supplementation for missing nutrients will be required. The sweetener aspartame, present in many diet foods and soft drinks, must also be avoided, as aspartame contains phenylalanine.

Throughout life, patients can use supplementary formulas, pills or specially formulated foods to acquire amino acids and other necessary nutrients that would otherwise be deficient in a low-phenylalanine diet. Some Phe is required for the synthesis of many proteins and is required for appropriate growth, but levels of Phe must be strictly controlled in PKU patients. Additionally, PKU patients must take supplements of tyrosine, which is normally derived from phenylalanine. Other supplements can include fish oil, to replace the long chain fatty acids missing from a standard Phe-free diet and improve neurological development, and iron or carnitine.

Another potential therapy for PKU is tetrahydrobiopterin (BH4), a cofactor for the oxidation of Phe that can reduce blood levels of Phe in certain patients. Patients who respond to BH4 therapy may also be able to increase the amount of natural protein that they can eat. However, BH4 therapy does not treat the fundamental problem of PAH deficiency and is suitable for only 10% of PKU patients. Therefore, an effective treatment of PKU with improved safety and dose reduction that does not elicit immune suppression is currently lacking.

rAAV PAH Vector Design

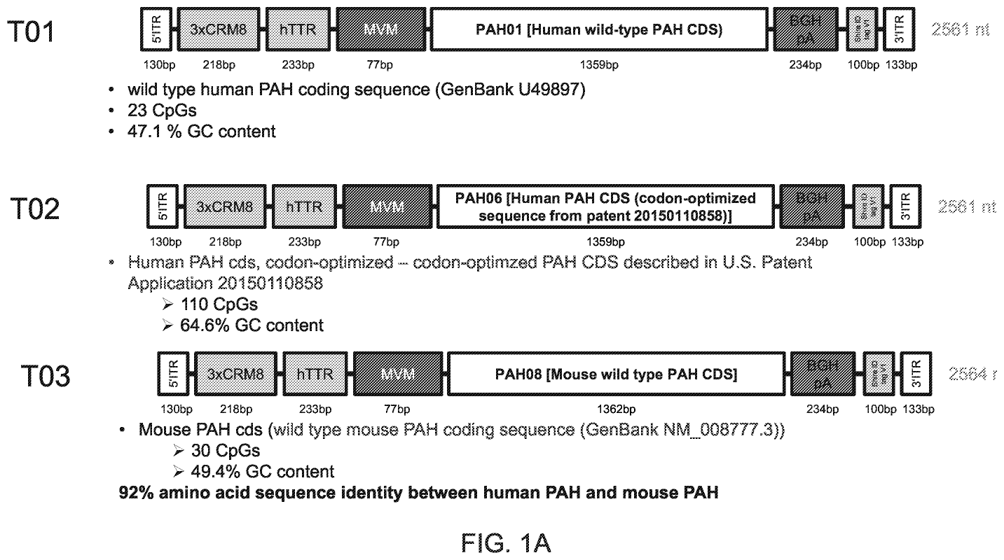

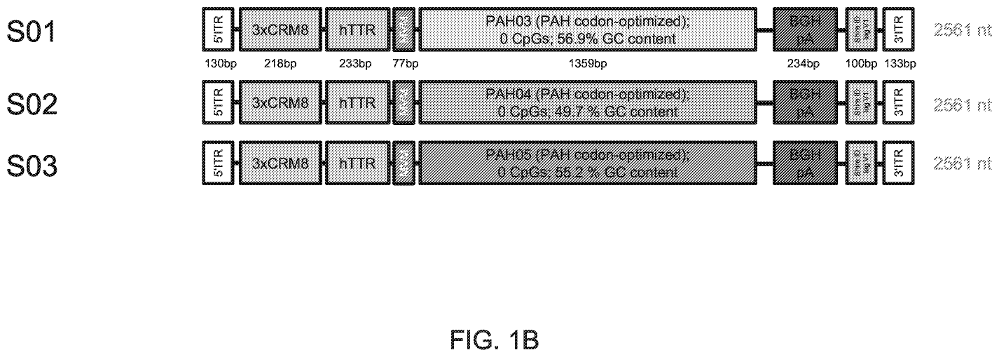

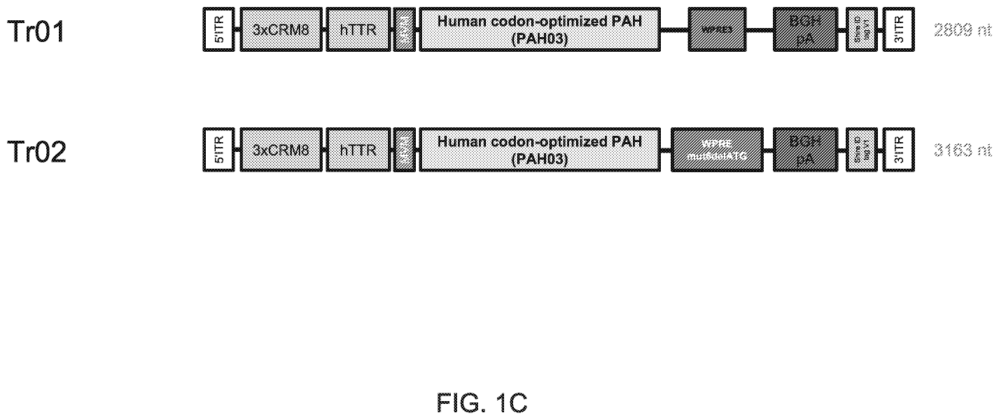

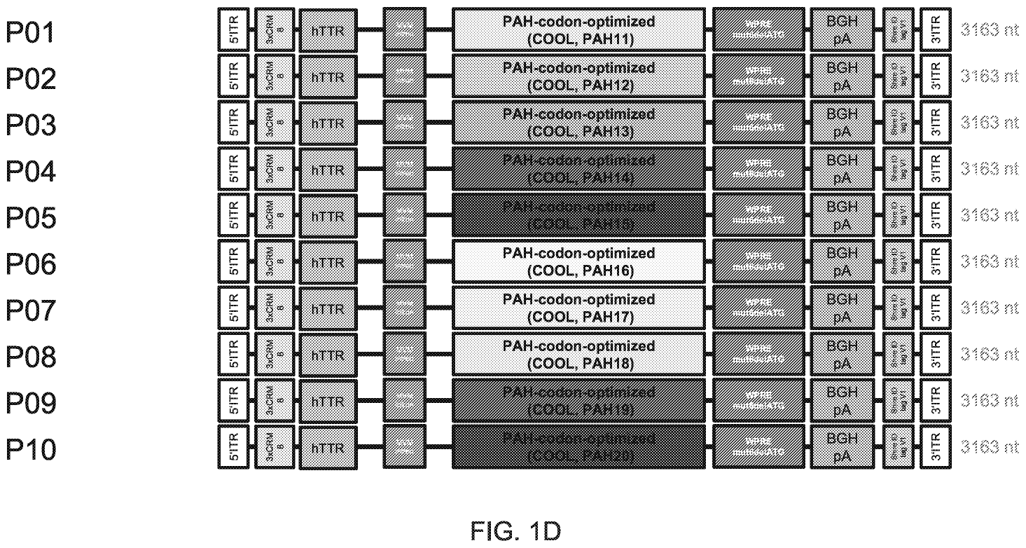

In some aspects, provided herewith is a recombinant adeno-associated virus (rAAV) vector encoding a phenylalanine hydroxylase (PAH) protein. A schematic that illustrates exemplary rAAV vectors of the present disclosure is illustrated in FIG. 1B. As shown in FIG. 1B, in some embodiments, an rAAV vector of the present disclosure comprises a liver specific promoter, a 5′ and a 3′ inverted terminal repeat (ITR), a cis-acting regulatory module (CRM), and an intron.

The PAH sequence of the vector can be a wild-type or a codon-optimized variant. Accordingly, in some embodiments, the rAAV vector comprises a wild-type PAH nucleotide sequence. In some embodiments, the rAAV vector comprises a codon-optimized PAH sequence.

A suitable PAH for the present invention is any protein or a portion of a protein that can substitute for at least partial activity of naturally-occurring phenylalanine hydroxylase (PAH) protein or rescue one or more phenotypes or symptoms associated with PAH-deficiency.

In some embodiments, a suitable PAH nucleotide sequence for the present invention comprises a PAH sequence encoding wt hPAH protein (GenBank U49897, the contents of which are incorporated herein by reference). In some embodiments, a suitable PAH nucleotide sequence for the present invention comprises a codon optimized nucleotide sequence encoding wild type human PAH protein. The naturally-occurring human PAH amino acid sequence is shown in Table 1:

TABLE 1
Human PAH
Human PAH (Amino Acid Seq.)

(SEQ ID NO:1)
MSTAVLENPGLGRKLSDFGQETSYIEDNCNQNGAISLIFSLKEEVGALA
KVLRLFEENDVNLTHIESRPSRLKKDEYEFFTHLDKRSLPALTNIIKIL
RHDIGATVHELSRDKKKDTVPWFPRTIQELDRFANQILSYGAELDADHP
GFKDPVYRARRKQFADIAYNYRHGQPIPRVEYMEEEKKTWGTVFKTLKS
LYKTHACYEYNHIFPLLEKYCGFHEDNIPQLEDVSQFLQTCTGFRLRPV
AGLLSSRDFLGGLAFRVFHCTQYIRHGSKPMYTPEPDICHELLGHVPLF
SDRSFAQFSQEIGLASLGAPDEYIEKLATIYWFTVEFGLCKQGDSIKAY
GAGLLSSFGELQYCLSEKPKLLPLELEKTAIQNYTVTEFQPLYYVAESF
NDAKEKVRNFAATIPRPFSVRYDPYTQRIEVLDNTQQLKILADSINSEI
GILCSALQKIK

Various kinds of promoters can be used in the rAAV vector described herein. These include, for example, ubiquitous, tissue-specific, and regulatable (e.g. inducible or repressible) promoters. In some embodiments, the promoter is a liver-specific promoter. Examples of liver-specific promoters are known in the art and include, for example, human transthyrethin promoter (hTTR), a-Antitrypsin promoter, human factor IX pro/liver transcription factor-responsive oligomers, LSP, and the basic albumin promoter. Liver specific promoters are described, for example, in Zhijian Wu et al., Molecular Therapy vol 16, no 2, February 2008, the contents of which are incorporated herein by reference.

In some embodiments, the promotor is a ubiquitous promoter. In some embodiments, the promoter is a chicken beta actin promoter.

In some embodiments, the rAAV vector contains additional enhancer or regulatory elements to promote transcription and/or translation of the mRNA (e.g., enhancer sequences, Kozak sequences, polyadenylation sequences, transcriptional termination sequences, IRES and the like). In some embodiments, the vector comprises a 5′ and a 3′ inverted terminal repeat (ITR). In some embodiments, the vector comprises one or more enhancer elements. In some embodiments, the vector comprises a poly(A) tail.

In some embodiments, the rAAV vector comprises one or more small elements, such as an intron. Various introns are known in the art. Suitable introns for the rAAV vector described herein include for example an MVM intron, a truncated F.IX intron, a chimeric β globin SD/immunoglobulin heavy chain SA intron, SV40 and/or an alpha globin 1^{st} intron. In some embodiments, the rAAV vector comprises an MVM intron. In some embodiments, the rAAV vector comprises an SV40 intron.

In some embodiments, the rAAV vector comprises woodchuck hepatitis virus post-transcriptional control element (WPRE). Various optimized or variant forms of WPRE are known in the art, and include WPRE3, WPREmut6delATG among others.

In some embodiments, the rAAV vector comprises a cis-actin regulatory module (CRM). Various kinds of CRM are suitable for use in the vectors described herein and include for example liver-specific CRM, neuronal-specific CRM and/or CRM8. In some embodiments, the vector includes more than one CRM. For example, in some embodiments, the vector comprises two, three, four, five or six CRM. In some embodiments, the vector comprises three CRM, for example three CRM8.

In some embodiments, the rAAV vector is sequence optimized to increase transcript stability, for more efficient translation, and/or to reduce immunogenicity. In some embodiments, the PAH is sequence optimized.

In some embodiments, the rAAV vector is an AAV1, AAV2, AAV3, AAV4, AAV5, AAV6, AAV7, AAV8, AAV9, AAV10, or AAV11 vector. In some embodiments, the rAAV vector is AAV1. In some embodiments, the rAAV vector is AAV2. In some embodiments, the rAAV vector is AAV3. In some embodiments, the rAAV vector is AAV4. In some embodiments, the rAAV vector is AAV5. In some embodiments, the rAAV vector is AAV6. In some embodiments, the rAAV vector is AAV7. In some embodiments, the rAAV vector is AAV8. In some embodiments, the rAAV vector is AAV9. In some embodiments, the rAAV vector is AAV10. In some embodiments, the rAAV vector is AAV11. In some embodiments, the rAAV vector is sequence optimized. In some embodiments, the rAAV capsid is modified. For example, in some embodiments, the rAAV8 capsid is modified.

Exemplary element sequences are shown in Table 2 below. In some embodiments, the rAAV vector comprises a rAAV vector element comprising a nucleotide sequence having at least 60%, 65%, 70%, 75%, 80%, 85%, 90%, 95% or 99% identity with a vector element sequence shown in Table 2. In some embodiments, the rAAV vector comprises a vector element nucleotide sequence identical to a vector element nucleotide sequence shown in Table 2.

TABLE 2
Exemplary rAAV Element Sequences
3xCRM8
(SEQ ID NO: 2)
GGGGGAGGCTGCTGGTGAATATTAACCAAGGTCACCCCAGTTATCGGAG
GAGCAAACAGGGGCTAAGTCCACCGGGGGAGGCTGCTGGTGAATATTAA
CCAAGGTCACCCCAGTTATCGGAGGAGCAAACAGGGGCTAAGTCCACCG
GGGGAGGCTGCTGGTGAATATTAACCAAGGTCACCCCAGTTATCGGAGG
AGCAAACAGGGGCTAAGTCCAC
hTTR promoter
(SEQ ID NO: 3)
AAATGACCTATTAAGAATATTTCATAGAACGAATGTTCCGATGCTCTAA
TCTCTCTAGACAAGGTTCATATTTGTATGGGTTACTTATTCTCTCTTTG
TTGACTAAGTCAATAATCAGAATCAGCAGGTTTGCAGTCAGATTGGCAG
GGATAAGCAGCCTAGCTCAGGAGAAGTGAGTATAAAAGCCCCAGGCTGG
GAGCAGCCATCACAGAAGTCCACTCATTCTTGGCAGG
MVM intron
(SEQ ID NO: 4)
CTAAGGTAAGTTGGCGCCGTTTAAGGGATGGTTGGTTGGTGGGGTATTA
ATGTTTAATTACCTTTTTTAGAGGCCTG
WPRE3
(SEQ ID NO: 5)
aatcaacctctggattacaaaatttgtgaaagattgactggtattctta
actatgttgctccttttacgctatgtggatacgctgctttaatgccttt
gtatcatgctattgcttcccgtatggctttcattttctcctccttgtat
aaatcctggttagttcttgccacggcggaactcatcgccgcctgccttg
cccgctgctggacaggggctcggctgttgggcactgacaattccgtggt
gtt
WPREmut6delATG
(SEQ ID NO: 6)
GATAATCAACCTCTGGATTACAAAATTTGTGAAAGATTGACTGGTATTC
TTAACTTTGTTGCTCCTTTTACGCTTTGTGGATACGCTGCTTTATTGCC
TTTGTATCTTGCTATTGCTTCCCGTTTGGCTTTCATTTTCTCCTCCTTG
TATAAATCCTGGTTGCTGTCTCTTTTTGAGGAGTTGTGGCCCGTTGTCA
GGCAACGTGGCGTGGTGTGCACTGTGTTTGCTGACGCAACCCCCACTGG
TTGGGGCATTGCCACCACCTGTCAGCTCCTTTCCGGGACTTTCGCTTTC
CCCCTCCCTATTGCCACGGCGGAACTCATCGCCGCCTGCCTTGCCCGCT
GCTGGACAGGGGCTGGGCTGTTGGGCACTGACAATTCCGTGGTGTTGTC
GGGGAAATCATCGTCCTTTCCTTGGCTGCTCGCCTGTGTTGCCACCTGG
ATTCTGCGCGGGACGTCCTTCTGCTACGTCCCTTCGGCCCTCAATCCAG
CGGACCTTCCTTCCCGCGGCCTGCTGCCGGCTCTGCGGCCTCTTCCGGG
TCTTCGCCTTCGCCCTCAGACGAGTCGGATCTCCCTTTGGGCCGCCTCC
CCGCATCGGACTAG
WPRE
(SEQ ID NO: 7)
ACCAGGTTCTGTTCCTGTTAATCAACCTCTGGATTACAAAATTTGTGAA
AGATTGACTGGTATTCTTAACTATGTTGCTCCTTTTACGCTATGTGGAT
ACGCTGCTTTAATGCCTTTGTATCATGCTATTGCTTCCCGTATGGCTTT
CATTTTCTCCTCCTTGTATAAATCCTGGTTGCTGTCTCTTTATGAGGAG
TTGTGGCCCGTTGTCAGGCAACGTGGCGTGGTGTGCACTGTGTTTGCTG
ACGCAACCCCCCTGGTTGGGGCATTGCCACCACCTGTCAGCTCCTTTCC
GGGACTTTCGCTTTCCCCCTCCCTATTGCCACGGCGGAACTCATCGCCG
CCTGCCTTGCCCGCTGCTGGACAGGGGCTCGGCTGTTGGGCACTGACAA
TTCCGTGGTGTTGTCGGGGAAGCTGACGTCCTTTCCATGGCTGCTCGCC
TGTGTTGCCACCTGGATTCTGCGCGGGACGTCCTTCTGCTACGTCCCTT
CGGCCCTCAATCCAGCGGACCTTCCTTCCCGCGGCCTGCTGCCGGCTCT
GCGGCCTCTTCCGCGTCTTCGCCTTCGCCCTCAGACGAGTCGGATCTCC
CTTTGGGCCGCCTCCCCGCCTGTTTCGCCTCGGGCTCCTCGAG
BGH pA
(SEQ ID NO: 8)
CCTAGAGCTCGCTGATCAGCCTCGACTGTGCCTTCTAGTTGCCAGCCAT
CTGTTGTTTGCCCCTCCCCCGTGCCTTCCTTGACCCTGGAAGGTGCCAC
TCCCACTGTCCTTTCCTAATAAAATGAGGAAATTGCATCGCATTGTCTG
AGTAGGTGTCATTCTATTCTGGGGGGTGGGGTGGGGCAGGACAGCAAGG
GGGAGGATTGGGAAGACAATAGCAGGCATGCTGGGGAA
3’ ITR
(SEQ ID NO: 9)
AGGAACCCCTAGTGATGGAGTTGGCCACTCCCTCTCTGCGCGCTCGCTC
GCTCACTGAGGCCGGGCGACCAAAGGTCGCCCGACGCCCGGGCTTTGCC
CGGGCGGCCTCAGTGAGCGAGCGAGCGCGCAGAGA
5’ ITR
(SEQ ID NO: 10)
CTGCGCGCTCGCTCGCTCACTGAGGCCGCCCGGGCAAAGCCCGGGCGTC
GGGCGACCTTTGGTCGCCCGGCCTCAGTGAGCGAGCGAGCGCGCAGAGA
GGGAGTGGCCAACTCCATCACTAGGGGTTCCT
Codon Optimized human PAH - 01
(SEQ ID NO: 11)
atgtccactgcggtcctggaaaacccaggcttgggcaggaaactctctg
actttggacaggaaacaagctatattgaagacaactgcaatcaaaatgg
tgccatatcactgatcttctcactcaaagaagaagttggtgcattggcc
aaagtattgcgcttatttgaggagaatgatgtaaacctgacccacattg
aatctagaccttctcgtttaaagaaagatgagtatgaatttttcaccca
tttggataaacgtagcctgcctgctctgacaaacatcatcaagatcttg
aggcatgacattggtgccactgtccatgagctttcacgagataagaaga
aagacacagtgccctggttcccaagaaccattcaagagctggacagatt
tgccaatcagattctcagctatggagcggaactggatgctgaccaccct
ggttttaaagatcctgtgtaccgtgcaagacggaagcagtttgctgaca
ttgcctacaactaccgccatgggcagcccatccctcgagtggaatacat
ggaggaagaaaagaaaacatggggcacagtgttcaagactctgaagtcc
ttgtataaaacccatgcttgctatgagtacaatcacatttttccacttc
ttgaaaagtactgtggcttccatgaagataacattccccagctggaaga
cgtttctcaattcctgcagacttgcactggtttccgcctccgacctgtg
gctggcctgctttcctctcgggatttcttgggtggcctggccttccgag
tcttccactgcacacagtacatcagacatggatccaagcccatgtatac
ccccgaacctgacatctgccatgagctgttgggacatgtgcccttgttt
tcagatcgcagctttgcccagttttcccaggaaattggccttgcctctc
tgggtgcacctgatgaatacattgaaaagctcgccacaatttactggtt
tactgtggagtttgggctctgcaaacaaggagactccataaaggcatat
ggtgctgggctcctgtcatcctttggtgaattacagtactgcttatcag
agaagccaaagcttctccccctggagctggagaagacagccatccaaaa
ttacactgtcacggagttccagcccctgtattacgtggcagagagtttt
aatgatgccaaggagaaagtaaggaactttgctgccacaatacctcggc
ccttctcagttcgctacgacccatacacccaaaggattgaggtcttgga
caatacccagcagcttaagattttggctgattccattaacagtgaaatt
ggaatcctttgcagtgccctccagaaaataaagtag
Codon Optimized human PAH - 03
(SEQ ID NO: 12)
atgagcactgctgtgctggagaaccctggcctgggcaggaaactgagtg
actttggccaggagaccagctacattgaggacaactgcaaccagaatgg
agccatcagcctgatcttcagcctgaaggaggaggtgggagccctggcc
aaggtgctgaggctgtttgaggagaatgatgtgaacctgacccacattg
agagcaggcccagcaggctgaagaaggatgagtatgagttcttcaccca
cctggacaagaggagcctgcctgccctgaccaacatcatcaagatcctg
aggcatgacattggagccacagtgcatgagctgagcagggacaagaaga
aggacacagtgccctggttccccaggaccatccaggagctggacaggtt
tgccaaccagatcctgagctatggagctgagctggatgctgaccaccct
ggcttcaaggaccctgtgtacagggccaggaggaagcagtttgctgaca
ttgcctacaactacaggcatggccagcccatccccagggtggagtacat
ggaggaggagaagaagacctggggcacagtgttcaagaccctgaagagc
ctgtacaagacccatgcctgctatgagtacaaccacatcttccccctgc
tggagaagtactgtggcttccatgaggacaacatcccccagctggagga
tgtgagccagttcctgcagacctgcacaggcttcaggctgaggcctgtg
gctggcctgctgagcagcagggacttcctgggaggcctggccttcaggg
tgttccactgcacccagtacatcaggcatggcagcaagcccatgtacac
ccctgagcctgacatctgccatgagctgctgggccatgtgcccctgttc
agtgacaggagctttgcccagttcagccaggagattggcctggccagcc
tgggagcccctgatgagtacattgagaagctggccaccatctactggtt
cacagtggagtttggcctgtgcaagcagggagacagcatcaaggcctat
ggagctggcctgctgagcagctttggagagctgcaatactgcctgagtg
agaagcccaagctgctgcccctggagctggagaagacagccatccagaa
ctacacagtgacagagttccagcccctgtactatgtggctgagagcttc
aatgatgccaaggagaaagtgaggaactttgctgccaccatccccaggc
ccttcagtgtgaggtatgacccctacacccagaggattgaggtgctgga
caacacccagcagctgaagatcctggctgacagcatcaacagtgagatt
ggcatcctgtgcagtgccctgcagaagatcaagtag
Codon Optimized human PAH - 04
(SEQ ID NO: 13)
atgtccactgcagtcctggagaacccaggcttgggcaggaaactctctg
actttggacaggagaccagctatattgaagacaactgcaaccaaaatgg
tgccatctccctgatcttctccctcaaagaggaagtgggtgcattggcc
aaagtcttgaggctttttgaggagaatgatgtcaacctgacccacattg
agtctagaccttctaggcttaagaaagatgagtatgagtttttcaccca
cttggataaaaggagcctgcctgctctgaccaacatcatcaagatcttg
aggcatgacattggtgccactgtccatgagctttccagggataagaaga
aagacacagtgccctggttcccaagaaccatccaagagctggacagatt
tgccaaccagatcctcagctatggagcagaactggatgctgaccaccct
ggcttcaaagatcctgtgtacagggcaagaagaaagcagtttgctgaca
ttgcctacaactacaggcatgggcagcccatccctagggtggaatacat
ggaggaggaaaagaaaacctggggcacagtgttcaagaccctgaagtcc
ttgtataaaacccatgcttgctatgagtacaaccacatctttccacttc
ttgagaagtactgtggcttccatgaagataacatcccccagctggagga
tgtgtctcagttcctgcagacctgcactggcttcaggctcaggcctgtg
gctggcctgctttcctctagagatttcttgggtggcctggccttcaggg
tcttccactgcacacagtacatcagacatggatccaagcccatgtatac
ccctgaacctgacatctgccatgagctgttgggacatgtgcccttgttt
tcagataggagctttgcccagttctcccaggagattggccttgcctctc
tgggtgcacctgatgaatacattgagaagctggccaccatctactggtt
cactgtggagtttgggctctgcaaacaaggagactccatcaaggcatat
ggtgctgggctcctgtcatcctttggtgaacttcagtactgcctttcag
agaagccaaagcttctccccctggagctggagaagacagccatccaaaa
ctacactgtcacagagttccagcccctctattatgtggcagagagcttc
aatgatgccaaggagaaagtcaggaactttgctgccaccatacctagac
ccttctcagtgaggtatgacccatacacccaaaggattgaggtcttgga
caacacccagcaacttaagatcttggctgattccatcaacagtgagatt
ggaatcctttgcagtgccctccagaaaatcaagtag
Codon Optimized human PAH - 05
(SEQ ID NO: 14)
atgtccactgctgtcctggagaacccaggcttgggcaggaagctctctg
actttgggcaggagaccagctacattgaggacaactgcaaccagaatgg
ggccatctccctgatcttctccctcaaggaggaagtgggggccctggcc
aaggtgttgaggctgtttgaggaaaatgatgtgaacctgacccacattg
agtccagaccctccaggctgaagaaggatgagtatgagttcttcaccca
cttggacaagaggagcctgcctgccctgaccaacatcatcaaaatcttg
aggcatgacattggggccactgtccatgagctgtccagggacaagaaaa
aggacacagtgccctggttccccagaaccatccaggagctggacagatt
tgccaaccagatcctcagctatggggctgagctggatgctgaccaccct
ggcttcaaggaccctgtgtacagggccagaagaaagcagtttgctgaca
ttgcctacaactacaggcatgggcagcccatccccagggtggagtacat
ggaggaagagaaaaagacctggggcacagtgttcaagaccctgaagtcc
ttatacaagacccatgcctgctatgagtacaaccacatcttccccctcc
tggagaagtactgtggcttccatgaggacaacatcccccagctggagga
tgtctcccagttcctgcagacctgcactggcttcaggctcaggcctgtg
gctggcctcctgtcctccagagacttcttgggaggcctggccttcaggg
tcttccactgcacacagtacatcagacatggctccaagcccatgtacac
ccctgagcctgacatctgccatgagctgttgggccatgtgcccttgttc
tcagacaggagctttgcccagttctcccaggagattggcctggcctccc
tgggagcccctgatgagtacattgagaagctggccaccatctactggtt
cactgtggagtttgggctctgcaagcagggggactccatcaaggcctat
ggggctgggctcctgtcatcctttggggagctgcaatactgcctgtcag
agaagcccaagctgctccccctggagctggagaagacagccatccagaa
ctacactgtcactgagttccagcccctctactatgtggctgagagtttc
aatgatgccaaggagaaagtgaggaactttgctgccaccatccctagac
ccttctcagtcaggtatgacccctacacccagaggattgaggtcttgga
caacacccagcagctgaagatcttggctgactccatcaacagtgagatt
ggcatcctgtgcagtgccctccagaagatcaagtag
Codon Optimized human PAH - 06
(SEQ ID NO: 15)
atgagcaccgccgtgctggagaaccccggcctgggccgcaagctgagcg
acttcggccaggagaccagctacatcgaggacaactgcaaccagaacgg
cgccatcagcctgatcttcagcctgaaggaggaggtgggcgccctggcc
aaggtgctgcgcctgttcgaggagaacgacgtgaacctgacccacatcg
agagccgccccagccgcctgaagaaggacgagtacgagttcttcaccca
cctggacaagcgcagcctgcccgccctgaccaacatcatcaagatcctg
cgccacgacatcggcgccaccgtgcacgagctgagccgcgacaagaaga
aggacaccgtgccctggttcccccgcaccatccaggagctggaccgctt
cgccaaccagatcctgagctacggcgccgagctggacgccgaccacccc
ggcttcaaggaccccgtgtaccgcgcccgccgcaagcagttcgccgaca
tcgcctacaactaccgccacggccagcccatcccccgcgtggagtacat
ggaggaggagaagaagacctggggcaccgtgttcaagaccctgaagagc
ctgtacaagacccacgcctgctacgagtacaaccacatcttccccctgc
tggagaagtactgcggcttccacgaggacaacatcccccagctggagga
cgtgagccagttcctgcagacctgcaccggcttccgcctgcgccccgtg
gccggcctgctgagcagccgcgacttcctgggcggcctggccttccgcg
tgttccactgcacccagtacatccgccacggcagcaagcccatgtacac
ccccgagcccgacatctgccacgagctgctgggccacgtgcccctgttc
agcgaccgcagcttcgcccagttcagccaggagatcggcctggccagcc
tgggcgcccccgacgagtacatcgagaagctggccaccatctactggtt
caccgtggagttcggcctgtgcaagcagggcgacagcatcaaggcctac
ggcgccggcctgctgagcagcttcggcgagctgcagtactgcctgagcg
agaagcccaagctgctgcccctggagctggagaagaccgccatccagaa
ctacaccgtgaccgagttccagcccctgtactacgtggccgagagcttc
aacgacgccaaggagaaggtgcgcaacttcgccgccaccatcccccgcc
ccttcagcgtgcgctacgacccctacacccagcgcatcgaggtgctgga
caacacccagcagctgaagatcctggccgacagcatcaacagcgagatc
ggcatcctgtgcagcgccctgcagaagatcaagtag
Codon Optimized human PAH - 08
(SEQ ID NO: 16)
atggcagctgttgtcctggagaacggagtcctgagcagaaaactctcag
actttgggcaggaaacaagttacatcgaagacaactccaatcaaaatgg
tgctgtatctctgatattctcactcaaagaggaagttggtgccctggcc
aaggtcctgcgcttatttgaggagaatgagatcaacctgacacacattg
aatccagaccttcccgtttaaacaaagatgagtatgagtttttcaccta
tctggataagcgtagcaagcccgtcctgggcagcatcatcaagagcctg
aggaacgacattggtgccactgtccatgagctttcccgagacaaggaaa
agaacacagtgccctggttcccaaggaccattcaggagctggacagatt
cgccaatcagattctcagctatggagccgaactggatgcagaccaccca
ggctttaaagatcctgtgtaccgggcgagacgaaagcagtttgctgaca
ttgcctacaactaccgccatgggcagcccattcctcgggtggaatacac
agaggaggagaggaagacctggggaacggtgttcaggactctgaaggcc
ttgtataaaacacatgcctgctacgagcacaaccacatcttccctcttc
tggaaaagtactgcggtttccgtgaagacaacatcccgcagctggaaga
tgtttctcagtttctgcagacttgtactggtttccgcctccgtcctgtt
gctggcttactgtcgtctcgagatttcttgggtggcctggccttccgag
tcttccactgcacacagtacattaggcatggatctaagcccatgtacac
acctgaacctgatatctgtcatgaactcttgggacatgtgcccttgttt
tcagatagaagctttgcccagttttctcaggaaattgggcttgcatcgc
tgggggcacctgatgagtacattgagaaactggccacaatttactggtt
tactgtggagtttgggctttgcaaggaaggagattctataaaggcatat
ggtgctgggctcttgtcatcctttggagaattacagtactgtttatcag
acaagccaaagctcctgcccctggagctagagaagacagcctgccagga
gtatactgtcacagagttccagcctctgtactatgtggccgagagtttc
aatgatgccaaggagaaagtgaggacttttgctgccacaatcccccggc
ccttctccgttcgctatgacccctacactcaaagggttgaggtcctgga
caatactcagcagttgaagattttagctgactccattaatagtgaggtt
ggaatcctttgccatgccctgcagaaaataaagtcatag
Codon Optimized human PAH - 11
(SEQ ID NO: 17)
atgagcacagcagtcctggagaaccctgggcttgggaggaaactgagtg
actttgggcaagagacctcctacattgaggataactgcaatcagaatgg
agccatcagcctcatcttctccctgaaagaggaggtgggggccctggcc
aaagtcctcagactctttgaggagaatgacgtgaacctgacccacattg
agagcagacccagtaggctcaagaaggatgagtatgaattcttcaccca
cctggacaagaggagcctgcctgccctcaccaacatcatcaaaatcctg
aggcatgacattggggccactgtccatgaactgtccagagacaagaaga
aagacacagtcccctggtttccaaggaccatccaagagctggaccgctt
tgccaaccagatcctctcctatggggctgagctggatgctgaccaccct
ggcttcaaggacccagtgtaccgggccagaaggaagcagtttgctgata
ttgcctacaattacagacatggccagcccatccccagagtggagtacat
ggaggaagagaaaaagacctggggcacagtgttcaaaaccctgaagtcc
ctctacaagacccatgcctgctatgagtacaaccacatctttcccctgc
tggagaagtactgtgggttccatgaggacaatatccctcagctggaaga
tgtgtcccagttcctgcagacctgcactggctttaggctgaggcctgtg
gctggcctgctcagctctagggacttcctgggggggctggccttcagag
tcttccactgcacccagtacatccgccatgggagcaagcccatgtacac
cccagagccagacatctgccatgagctgctgggccatgtgcccctcttc
tcagacagaagctttgcccagttcagccaagaaattggactggcctccc
tgggtgcccctgatgaatacatagaaaagctggctaccatctactggtt
cacagtggaatttggcctctgcaagcaaggggactccatcaaggcctat
ggagctgggctcctgagctcctttggagagctccaatactgcctgtctg
agaagcccaagctcctgcccctggagcttgagaagacagccatccagaa
ctacactgtgactgagttccagcctctgtactacgtggcagagtccttc
aatgatgccaaggagaaggtgaggaactttgcagccaccattcctaggc
ccttctctgtgaggtatgacccctacacacagaggattgaagtgctgga
caacacccagcagctcaagatcctggcagacagcatcaactcagagatt
ggcatcctgtgctctgccctccagaagattaagtag
Codon Optimized human PAH - 12
(SEQ ID NO: 18)
atgagcacagcagtgttggagaaccctgggcttgggaggaaactgtctg
actttggacaagagacctcctacatagaagacaactgcaatcagaatgg
agccatctccctcatcttcagcctcaaggaagaggtgggggccctggcc
aaagtcctgaggctctttgaggagaatgacgtgaacctgacccacattg
agagtaggccctcccggctgaagaaggatgaatatgaattcttcaccca
cctggataagaggagcctgcctgccctcaccaacatcatcaaaatcctg
aggcatgacattggggccactgtgcatgagctctcaagggacaagaaga
aagacacagtcccctggttccctaggaccatccaagagctggaccgctt
tgccaaccagatcctctcctatggggctgagctggatgctgaccaccct
ggcttcaaggacccagtgtaccgggccagaaggaagcagtttgcagata
ttgcctacaattacagacatggccagcccatcccaagggtggaatacat
ggaggaagaaaaaaagacctggggcactgtcttcaaaaccctgaagagc
ctgtacaagacccatgcctgctatgagtacaaccacatctttcccctgc
tggagaagtactgtgggttccatgaggacaatatccctcagctggagga
tgtgtcccagttcctccagacctgcactggctttaggctgaggcctgtg
gctggcctgctgtccagcagagacttcctggggggcttggccttcagag
tgttccactgcacccagtacatccgccatgggagcaagcccatgtacac
cccagagccagacatctgccatgaactgctgggccatgtgcccctcttc
agtgacagaagctttgcccagttctcccaagaaattggactggcctccc
tgggtgcccctgatgagtacattgagaagctggccaccatctactggtt
tacagtggagtttggcctctgcaagcaaggggactccatcaaggcctat
ggagctgggctgctcagctcctttggggagctgcaatactgcctgagtg
agaaacccaagctcctgcccctggagctggaaaagacagccatccagaa
ctacacagtcacagagttccagcctctctactatgtggcagagagcttc
aatgatgccaaggagaaggtgaggaactttgctgccaccatccccagac
ccttctctgtgaggtatgacccctacactcagaggattgaagtgctgga
caacacccagcagctcaagattctggctgatagcatcaactcagagatt
ggcatcctgtgctctgccctgcagaagatcaagtag
Codon Optimized human PAH - 13
(SEQ ID NO: 19)
atgagcacagcagtgttggagaaccctgggcttgggaggaaactgtctg
actttgggcaagagacctcctacattgaggacaactgcaatcagaatgg
ggccatcagcctcatcttttccctgaaagaggaggtgggggccctggcc
aaagtcctgaggctctttgaggagaatgacgtgaacctgacccacattg
agagtaggccctctaggctgaagaaggatgaatatgaattcttcaccca
cctggacaagaggagcctgcctgccctcaccaacatcatcaaaatcctg
aggcatgacattggagccactgtccatgagctgtccagagataagaaga
aagacacagtcccctggtttccccgcaccatccaagagcttgaccgctt
tgccaaccagatcctgagctatggggcagagctggatgctgaccaccct
ggcttcaaggacccagtgtacagagccagaaggaagcagtttgctgata
ttgcctacaattaccgccatggacagcccatcccaagggtggaatacat
ggaggaagagaaaaagacctggggcacagtcttcaaaaccctgaagtcc
ctctacaagacccatgcctgctatgagtacaaccacatcttccccctgc
tggagaagtactgtggcttccatgaagacaatatccctcagctggagga
tgtgtcccagttcctgcagacctgcactgggttccgcctgagacctgtg
gctgggctcctctccagcagagacttcctggggggcttggccttcagag
tgttccactgcacccagtacatcagacatggcagcaagcccatgtacac
cccagagcctgacatctgccatgagctcctgggccatgtgcccctcttc
agtgacagaagctttgcccagttctcccaagaaattgggctggcctccc
tgggtgcccctgatgagtacatagaaaagctggctaccatctactggtt
cacagtggagtttggcctgtgcaagcaaggggacagcatcaaggcctat
ggagctggcctgctcagctcctttggagagctccaatactgcctgagtg
agaagcccaagctcctgcccctggaactggagaagacagccatccagaa
ctacactgtgactgagttccagcctctgtactatgtggctgagagcttc
aatgatgccaaggagaaggtgaggaactttgcagccaccatccccagac
ccttctctgtgaggtatgacccctacacacagaggattgaagtgctgga
caacacccagcagctcaagatcctggcagactccatcaactcagagatt
ggcatcctctgcagtgccctccagaagattaagtag
Codon Optimized human PAH - 14
(SEQ ID NO: 20)
atgagcacagcagtcctggagaaccctgggcttgggaggaaactgtctg
actttgggcaagagacctcctacatagaagacaactgcaatcagaatgg
agccatcagcctcatcttctccctgaaagaggaggtgggggccctggca
aaggtgctgaggctgtttgaggagaatgatgtgaacctgacccacattg
aatctaggccctcccggctgaagaaggatgaatatgaattcttcaccca
cctggacaagaggagcctgcctgccctcaccaacatcatcaaaatcctg
aggcatgacattggggccactgtccatgaactgtccagagataagaaga
aagacacagtcccctggttccctaggaccatccaagagctggacagatt
tgccaaccagatcctctcctatggggctgagctggatgctgaccaccct
ggcttcaaggaccctgtctatagggccagaaggaagcagtttgctgata
ttgcctacaattacagacatggccagcccatcccaagggtggaatacat
ggaggaagagaaaaagacctggggcacagtcttcaaaaccctgaagtcc
ctctacaagacccatgcctgctatgagtacaaccacatcttccccctgc
tggaaaagtactgtgggttccatgaggacaatatccctcagctggagga
tgtctcccagttcctccagacctgcactggcttccgcctgagacctgtg
gctgggctcctgagcagcagagacttcctgggggggctggccttcagag
tgttccactgcacacagtacatccgccatgggagcaagcccatgtacac
cccagagccagacatctgccatgagctgctgggccatgtgcccctcttc
agtgaccgcagctttgcccagttcagccaagaaattggactggcctccc
tgggtgcccctgatgagtacattgagaagctggccaccatctactggtt
tactgtggagtttggcctctgcaagcaaggggacagcatcaaggcctat
ggagctggcctgctcagctcctttggagagctccaatactgcctgagtg
agaaacccaagctcctgcccctggagctggagaagacagccatccagaa
ctacacagtgacagagttccagcctctgtactacgtggcagagagcttc
aatgatgccaaggagaaggtcagaaactttgcagccaccatccccagac
ccttctctgtgaggtatgacccctacactcagaggattgaggtcttgga
caacacccagcagctcaagattctggctgactccatcaactcagagat
tggcatcctgtgctctgccctgcagaagatcaagtaa
Codon Optimized human PAH - 15
(SEQ ID NO: 21)
atgagcacagcagtcctggagaaccctgggcttgggaggaagctctctg
actttgggcaagagacctcctacatagaagacaactgcaatcagaatgg
agccatcagcctcatcttctccctcaaggaagaagtgggggccctggca
aaggtcctgaggctctttgaggagaatgatgtcaacctgacccacattg
agtctaggcccagcagactgaagaaggatgaatatgaattcttcaccca
cctggacaagaggagcctgcctgccctcaccaacatcatcaaaatcctg
aggcatgacattggggccactgtccatgagctctccagagacaaaaaga
aggacaccgtcccctggttccctaggaccatccaagaactggaccgctt
tgccaaccagatcctctcctatggggcagagctggatgctgaccaccct
ggcttcaaagacccagtgtacagagccagaaggaagcagtttgcagata
ttgcctacaattaccgccatggacagcccatcccaagggtggaatacat
ggaagaggagaagaaaacctggggcacagtgttcaagaccctgaagagc
ctgtacaagacccatgcctgctatgagtacaaccacatctttcccctgc
tggaaaagtactgtgggttccatgaggacaatatccctcagctggagga
tgtgtcccagttcctccagacctgcactggctttaggctgaggcctgtg
gctggcctgctgtccagtagggacttcctggggggcttggccttcagag
tcttccactgcacccagtacatcagacatggcagcaagcccatgtacac
cccagagcctgacatctgccatgaactcctgggccatgtgcccctcttc
agtgacagatcctttgcccagttcagccaagagatcgggctggcctccc
tgggtgcccctgatgagtacattgaaaaactggccaccatctactggtt
tactgtggagtttggcctctgcaagcaaggggacagcatcaaggcctat
ggagctgggctcctcagcagctttggagagctccaatactgcctgtctg
agaaacccaagctgctccccctggagctggagaagacagccatccagaa
ctacacagtcacagagttccagcctctctactacgtggctgagagcttc
aatgatgccaaggagaaggtgaggaactttgctgccaccatccccagac
ccttctctgtgaggtatgacccctacactcagaggattgaggtgctgga
caacacccagcagctcaagattctggctgactccatcaactcagagatt
ggcatcctgtgctctgccctgcagaagatcaagtaa
Codon Optimized human PAH - 16
(SEQ ID NO: 22)
atgagcacagcagtgttggagaaccctgggcttgggaggaaactgtctg
actttgggcaagagacctcctacattgaagacaactgcaatcagaatgg
agccatcagcctcatcttttccctgaaagaggaggtgggggccctggcc
aaagtcctgaggctctttgaggagaatgacgtgaacctgacccacattg
agagtaggcccagcagactgaagaaggatgaatatgaattcttcaccca
cctggacaagaggagcctgcctgccctcaccaacatcatcaaaatcctg
aggcatgacattggggccactgtccacgagctctcaagggacaagaaga
aagacacagtcccctggtttccaaggaccatccaagagcttgaccgctt
tgccaaccagatcctctcctatggggctgagctggatgctgaccaccct
ggctttaaggacccagtgtatagggccagaaggaagcagtttgctgata
ttgcctacaactacagacatggccagcccatcccaagggtggaatacat
ggaggaagagaaaaagacctggggcacagtgttcaagaccctgaagagc
ctgtacaagacccatgcctgctatgagtacaaccacatcttccccctgc
tggaaaagtactgtggcttccatgaggacaatatccctcagctggagga
tgtgtcccagttcctccagacctgcactgggtttaggctgaggcctgtg
gctgggctcctcagcagccgggacttcctgggggggctggccttcagag
tcttccactgcacccagtacatccgccatgggagcaagcccatgtacac
cccagagccagacatctgccatgaactgctgggccatgtgcccctcttc
agtgacagatcctttgcccagttctcccaagaaattggactggcctccc
tgggtgcccctgatgagtacatagagaagctggctaccatctactggtt
cacagtggagtttggcctctgcaagcaaggggactccatcaaggcctat
ggagctggcctgctgtccagctttggagagctgcagtattgcctgagtg
agaaacccaagctcctgcccctggagctggagaagacagccatccagaa
ctatactgtgactgagttccagcctctctactatgtggcagagagcttc
aatgatgccaaggagaaggtgaggaactttgcagccaccatccccagac
ccttctctgtgaggtatgacccctacactcagaggatagaagtgctgga
caacacccagcagctcaagatcctggcagacagcatcaactcagagatt
ggcatcctgtgctctgccctccagaagattaagtaa
Codon Optimized human PAH - 17
(SEQ ID NO: 23)
atgtccactgctgttctggagaaccctggactggggaggaagctctctg
actttgggcaagagacctcctacattgaggacaactgcaaccagaatgg
ggccatcagcctcatcttctccctgaaagaggaggtgggggccctggcc
aaggtcctgaggctctttgaggagaatgatgtgaacctgactcacattg
agagccggcccagtaggctgaagaaggatgagtatgaattcttcaccca
cctggataagaggagcctgcctgccctgaccaacatcatcaaaatcctg
aggcatgacattggagccactgtccacgagctctcaagggacaagaaga
aagacactgtgccctggtttccaaggaccatccaagagctggacagatt
tgccaatcagatcctctcctatggggcagagctggatgctgaccaccct
ggcttcaaggaccctgtctatagggctaggaggaagcagtttgcagata
ttgcctacaattaccgccatggacagcccatcccaagggtggagtacat
ggaggaagagaaaaagacctggggcacagtcttcaaaaccctgaagtcc
ctctacaagacccacgcctgctatgagtacaaccacatcttccccctgc
tggagaagtactgtgggttccatgaagacaatatcccccagcttgagga
tgtctcccagttcctccagacctgtactggctttaggctgaggcctgtg
gctgggctcctgtccagcagagacttcctggggggcttggccttcagag
tgttccactgcacacagtacatcagacatggcagcaagcccatgtacac
cccagagccagacatctgccatgagctgctgggccatgtccccctcttc
agtgaccgcagctttgcccagttcagccaagaaattgggctggcctccc
tgggggctcctgatgaatacatagagaagctggccaccatctactggtt
cacagttgagtttggcctctgcaagcaaggggacagcatcaaggcctat
ggagctggcctgctcagctcctttggagagctgcagtattgtctgtctg
agaagcccaagctcctgcccctggagctggaaaagacagccatccagaa
ctacacagtgacagagttccagcctctgtactatgtggctgagtccttc
aatgatgccaaggagaaggtgaggaattttgctgccaccattcccagac
ccttctctgtgaggtatgacccctacactcagaggattgaagtgctgga
caacacccagcagctcaagatcctggcagactccatcaactcagagatt
ggcatcctgtgttctgccctccagaagatcaagtga
Codon Optimized human PAH - 18
(SEQ ID NO: 24)
atgagcacagcagttctggagaaccctggactggggaggaagctgtctg
actttggacaagagacctcctacatagaggacaactgcaatcagaatgg
agccatcagcctcatcttcagcctcaaggaggaagtgggggccctggcc
aaggtcctgaggctgtttgaggagaatgatgtgaacctgactcacattg
agagtaggccctcaaggctcaagaaggatgagtatgagttcttcaccca
cctggataagaggtccctgcctgccctgaccaacatcatcaaaatcctg
cgccatgacattggggccacagtgcacgagctctcaagggacaagaaga
aagacacagtcccctggtttccccgcaccatccaagagctggacagatt
tgccaaccagatcctctcctatggggctgagctggatgctgaccaccct
ggctttaaggacccagtgtatagggccagaaggaagcagtttgctgata
ttgcctacaattacagacatggccagcccatccccagagttgagtacat
ggaggaagagaaaaagacctggggcactgttttcaagaccctgaagtcc
ctctacaagacccacgcctgctatgagtacaaccacatcttcccactgc
tggaaaagtactgtggcttccatgaggacaatatccctcagctggagga
tgtctcccagttcctccagacctgtactgggtttaggctgaggcctgta
gctggcctgctcagctctagggacttcctgggagggctggccttccggg
tcttccactgcacccagtacatcagacatgggagcaagcccatgtacac
cccagagccagacatctgccatgagctgctgggccatgtgcccctcttc
tcagataggtcctttgcccagttctcccaagagataggcctggcatccc
tgggtgcccctgatgagtacattgagaagctggccaccatctactggtt
cactgtggagtttggcctctgcaagcaaggggacagcatcaaggcctat
ggagctgggctcctgtccagctttggggagctgcagtattgtctgagtg
agaagcccaagctcctgccactggagctggagaagacagccatccagaa
ctacacagtcacagagttccagcctctgtactatgtggcagagagcttc
aatgatgccaaggagaaggtgaggaattttgcagccaccatcccaagac
ccttctctgtgaggtatgacccctacactcagaggattgaggtgctgga
caacacccagcagctcaagattctggctgactccatcaactcagagatt
ggcatcctgtgttctgccctccagaagatcaagtga
Codon Optimized human PAH - 19
(SEQ ID NO: 25)
atgagcacagctgtgctggagaaccctgggcttggaaggaagctcagtg
actttggccaagagacctcctacattgaggacaactgcaatcagaatgg
agccatcagcctcatcttttccttgaaggaagaagtgggggccttggcc
aaagtcctgaggctgtttgaggagaatgacgtcaacctgactcacattg
aatctaggccttcaaggctcaagaaggatgagtatgaattcttcaccca
cctggacaagaggagcctgcctgctctgaccaacatcatcaaaatcttg
aggcatgacattggagcaacagtccacgagcttagcagagacaaaaaga
aagacaccgtgccctggttcccaagaaccattcaagagttggataggtt
tgccaaccagatcctctcctatggggctgagctggatgctgaccaccct
ggctttaaggaccctgtgtatagagccagaagaaagcagtttgctgata
ttgcctacaattacagacatggacagcccatccccagagtggagtacat
ggaggaagagaaaaaaacctggggcactgtcttcaagaccctgaaatct
ctgtacaagacacatgcctgctatgagtacaaccacatcttccctctgt
tggagaagtactgtggcttccatgaagataacattccccagcttgagga
tgtgtctcaatttctccagacctgcactggattcagactcagaccagtg
gctggcctgctgtccagtagggacttcctgggaggactggcctttaggg
tgttccactgcacacagtacatcagacacggcagcaagcccatgtacac
accagagccagacatctgccatgagctcctgggccatgtccccctcttc
tctgacagatcctttgcccagttctcccaagaaattggtctggcttccc
tgggtgcccctgatgaatatatagaaaagctggccaccatctactggtt
tacagtggaatttgggctctgcaaacaaggagactccattaaggcctat
ggagctgggctgctcagcagctttggagagctgcaatactgcctgtctg
aaaaacccaagcttctgcccctggaactggagaaaacagcaatccagaa
ctacactgtgactgagttccagcctctctactacgtggcagagagcttc
aatgatgccaaggagaaggtgagaaactttgcagccactatcccaaggc
ccttcagtgttagatatgacccctacacccagaggattgaggtgcttga
caatactcagcagctgaagattctggcagattccatcaactcagagatt
ggcatcctgtgttctgccctgcagaagatcaagtaa
Codon Optimized human PAH - 20
(SEQ ID NO: 27)
atgtccactgctgtgttggagaaccctggacttggcagaaaactgagtg
actttgggcaagagacctcctacattgaagataactgcaatcagaatgg
agccatttccctcatcttctccctgaaggaagaagtgggggccctggcc
aaagtcctgcgcctgtttgaggagaatgacgtcaacctgacccacatcg
aatctaggccttcaaggttgaagaaggatgaatatgagttcttcacaca
cctggataagaggagcctgcctgccctcaccaacatcatcaaaatcttg
aggcatgacattggagcaacagtccacgagctgagcagagacaagaaga
aagacaccgtcccctggtttcccagaaccatccaagaacttgaccgctt
cgccaaccagatcctgtcctatggcgcagagcttgatgctgaccaccct
gggttcaaggacccagtgtacagagccagaaggaagcagtttgcagata
ttgcctacaattaccgccatggccagcccatcccaagagtggagtacat
ggaggaagagaaaaagacctggggcactgtgttcaaaaccctgaaaagc
ctctacaagactcacgcctgctatgaatacaaccacattttcccactgc
ttgagaagtactgtggcttccatgaggacaatatcccccagctggagga
tgtgtctcaatttctgcagacctgcactggctttcggctgagacctgtg
gccggcctcctcagcagccgggacttcctgggaggcttggccttcagag
tcttccactgcacacagtatatcagacatggaagcaagcccatgtacac
tccagagccagacatctgccatgaactgctgggccatgtgcccctcttc
tctgaccggagctttgcccagttcagccaagagattggccttgcctctc
tgggggctcctgatgagtacatcgagaagctggctaccatctactggtt
caccgtggaatttggcctgtgcaaacaaggagactccatcaaggcctat
ggagctgggctgctctcctcctttggagagctccagtactgcctgtctg
aaaaacccaagctcctgcccctggagctggaaaagacagccatccagaa
ctacacagtgacagaattccagcctctgtactacgtggctgagagcttc
aatgatgccaaggagaaggtgagaaactttgctgccaccattcctcggc
ccttttctgtgcgctatgacccctacacccaaagaattgaggtgctgga
caacacccagcagctcaagattctggcagacagcatcaactcagagatc
ggcatcctctgctccgcccttcagaagatcaagtaa

In some embodiments, the rAAV PAH vector comprises a codon-optimized PAH nucleotide having at least 70%, 75%, 80%, 85%, 90%, 95% or 99% identity with one of SEQ ID Nos: 11-27. Accordingly, in some embodiments, the rAAV PAH vector comprises a codon-optimized PAH nucleotide having at least 70% identity with one of SEQ ID Nos.: 11-27. In some embodiments, the rAAV PAH vector comprises a codon-optimized PAH nucleotide having at least 75% identity with one of SEQ ID Nos.: 11-27. In some embodiments, the rAAV PAH vector comprises a codon-optimized PAH nucleotide having at least 80% identity with one of SEQ ID Nos.: 11-27. In some embodiments, the rAAV PAH vector comprises a codon-optimized PAH nucleotide having at least 85% identity with one of SEQ ID Nos.: 11-27. In some embodiments, the rAAV PAH vector comprises a codon-optimized PAH nucleotide having at least 90% identity with one of SEQ ID Nos.: 11-27. In some embodiments, the rAAV PAH vector comprises a codon-optimized PAH nucleotide having at least 95% identity with one of SEQ ID Nos.: 11-27. In some embodiments, the rAAV PAH vector comprises a codon-optimized PAH nucleotide having at least 99% identity with one of SEQ ID Nos.: 11-27. In some embodiments, the rAAV PAH vector comprises a codon-optimized PAH nucleotide sequence identical to one of SEQ ID Nos: 11-27.

Use of rAAV Vectors that Encode PAH for Treatment of Disease

Described herein are methods of treating a disease associated with PAH enzyme deficiency. Accordingly, in some embodiments, the rAAV vectors described herein are suitable for treating a subject that has a PAH deficiency, such as phenylketonuria (PKU). The method of treating includes administering to the subject in need thereof a recombinant adeno-associated virus (rAAV) vector as described herein.

The rAAV vector described herein can be used to treat any disease associated with PAH deficiency or disorder.

In some embodiments, the rAAV vector remains episomal following administration to a subject in need thereof. In some embodiments, the rAAV vector does not remain episomal following administration to a subject in need thereof. For example, in some embodiments, the rAAV vector integrates into the genome of the subject. Such integration can be achieved, for example, by using various gene-editing technologies, such as, zinc finger nucleases (ZFNs), Transcription activator-like effector nucleases (TALENS), ARCUS genome editing, and/or CRISPR-Cas systems.

In some embodiments, a pharmaceutical composition comprising a rAAV vector described herein is used to treat subjects in need thereof. The pharmaceutical composition containing a rAAV vector of the invention contains a pharmaceutically acceptable excipient, diluent or carrier. Examples of suitable pharmaceutical carriers are well known in the art and include phosphate buffered saline solutions, water, emulsions, such as oil/water emulsions, various types of wetting agents, sterile solutions and the like. Such carriers can be formulated by conventional methods and are administered to the subject at a therapeutically effective amount.

The rAAV vector is administered to a subject in need thereof via a suitable route. In some embodiments, the rAAV vector is administered by intravenous, intraperitoneal, subcutaneous, or intradermal administration. In some embodiments, the rAAV vector is administered intravenously. In some embodiments, the intradermal administration comprises administration by use of a “gene gun” or biolistic particle delivery system. In some embodiments, the rAAV vector is administered via a non-viral lipid nanoparticle. For example, a composition comprising the rAAV vector may comprise one or more diluents, buffers, liposomes, a lipid, a lipid complex. In some embodiments, the rAAV vector is comprised within a microsphere or a nanoparticle, such as a lipid nanoparticle.

In some embodiments, functional PAH is detected in the subject. Various manners of detecting PAH can be used and can include, for example, tissue sampling (including biopsy) and screening for the presence of PAH. In some embodiments, functional PAH is detectable in the subject at about 2 to 6 weeks post administration of the rAAV vector. In some embodiments, functional PAH is detectable in the subject at about 2 weeks post administration of the rAAV vector. In some embodiments, functional PAH is detectable in the subject at about 3 weeks post administration of the rAAV vector. In some embodiments, functional PAH is detectable in the subject at about 4 weeks post administration of the rAAV vector. In some embodiments, functional PAH is detectable in the subject at about 5 weeks post administration of the rAAV vector. In some embodiments, functional PAH is detectable in the subject at about 6 weeks post administration of the rAAV vector. In some embodiments, functional PAH is detectable in hepatocytes of the subject at about 2 to 6 weeks post administration of the rAAV vector. In some embodiments, functional PAH is detectable in hepatocytes of the subject greater than 7 weeks post administration of the rAAV vector.

In some embodiments, functional PAH is detectable in the subject at least 3 months, 6 months, 12 months, 2 years, 3 years, 4 years, 5 years, 6 years, 7 years, 8 years, 9 years, or 10 years after administration of the rAAV vector. Accordingly, in some embodiments, functional PAH is detectable in the subject at least 3 months after administration of the rAAV vector. In some embodiments, functional PAH is detectable in the subject at least 6 months after administration of the rAAV vector. In some embodiments, functional PAH is detectable in the subject at least 12 months after administration of the rAAV vector. In some embodiments, functional PAH is detectable in the subject at least 2 years after administration of the rAAV vector. In some embodiments, functional PAH is detectable in the subject at least 3 years after administration of the rAAV vector. In some embodiments, functional PAH is detectable in the subject at least 4 years after administration of the rAAV vector. In some embodiments, functional PAH is detectable the subject at least 5 years after administration of the rAAV vector. In some embodiments, functional PAH is detectable in the subject at least 6 years after administration of the rAAV vector. In some embodiments, functional PAH is detectable in the subject at least 7 years after administration of the rAAV vector. In some embodiments, functional PAH is detectable in the subject at least 8 years after administration of the rAAV vector. In some embodiments, functional PAH is detectable in the subject at least 9 years after administration of the rAAV vector. In some embodiments, functional PAH is detectable in the subject at least 10 years after administration of the rAAV vector. In some embodiments, functional PAH is detectable in the subject for the remainder of the subject's life following administration of the rAAV vector.

In some embodiments, the administered rAAV comprising PAH results in the production of active PAH in a therapeutically effective amount.

In some embodiments, the administered rAAV comprising PAH results in the reduction of phenylalanine (Phe) in the subject. In some embodiments, the reduction of Phe is detected in plasma of the subject. In some embodiments, the reduction of Phe is detected in central nervous system (CNS). In some embodiments, the reduction of Phe is detected in brain tissue of the subject. In some embodiments, the reduction of Phe is detected in liver tissue of the subject. In some embodiments, the administered rAAV comprising PAH reduces Phe in the subject by about 95%, 90%, 85%, 80%, 75%, 70%, 65%, 60%, 55%, 50%, 45%, 40%, 35%, 30%, 25%, 20%, 15%, or about 10% in comparison to the subject's baseline Phe level prior to administering the rAAV comprising PAH. Accordingly, in some embodiments, the administered rAAV comprising PAH reduces Phe in the subject by about 95%. In some embodiments, the administered rAAV comprising PAH reduces Phe in the subject by about 90%. In some embodiments, the administered rAAV comprising PAH reduces Phe in the subject by about 85%. In some embodiments, the administered rAAV comprising PAH reduces Phe in the subject by about 80%. In some embodiments, the administered rAAV comprising PAH reduces Phe in the subject by about 75%. In some embodiments, the administered rAAV comprising PAH reduces Phe in the subject by about 70%. In some embodiments, the administered rAAV comprising PAH reduces Phe in the subject by about 65%. In some embodiments, the administered rAAV comprising PAH reduces Phe in the subject by about 60%. In some embodiments, the administered rAAV comprising PAH reduces Phe in the subject by about 55%. In some embodiments, the administered rAAV comprising PAH reduces Phe in the subject by about 50%. In some embodiments, the administered rAAV comprising PAH reduces Phe in the subject by about 45%. In some embodiments, the administered rAAV comprising PAH reduces Phe in the subject by about 40%. In some embodiments, the administered rAAV comprising PAH reduces Phe in the subject by about 35%. In some embodiments, the administered rAAV comprising PAH reduces Phe in the subject by about 30%. In some embodiments, the administered rAAV comprising PAH reduces Phe in the subject by about 25%. In some embodiments, the administered rAAV comprising PAH reduces Phe in the subject by about 20%. In some embodiments, the administered rAAV comprising PAH reduces Phe in the subject by about 15%. In some embodiments, the administered rAAV comprising PAH reduces Phe in the subject by about 10%.

In some embodiments, the administered rAAV comprising PAH results in the increase of non-Phe large neutral amino acids (LNAAs) in the subject. Without wishing to be bound by theory, various modes of action may result in the increase of LNAAs in the subject including, for example, increased production of LNAAs, increased transport or trafficking of LNAAs, and/or increased stability of LNAAs. In some embodiments, the increase of non-Phe LNAAs is detected in plasma of the subject. In some embodiments, the increase of non-Phe LNAAs is detected in central nervous system (CNS). In some embodiments, the increase of non-Phe LNAAs is detected in brain tissue of the subject. In some embodiments, the increase of non-Phe LNAAs is detected in liver tissue of the subject. In some embodiments the non-Phe LNAA is tyrosine. In some embodiments, the non-Phe LNAA is tryptophan. In some embodiments, the non-Phe LNAA is valine. In some embodiments, the non-Phe LNAA is isoleucine. In some embodiments, the non-Phe LNAA is methionine. In some embodiments, the non-Phe LNAA is threonine. In some embodiments, the non-Phe LNAA is leucine. In some embodiments, the non-Phe LNAA is histidine.

In some embodiments, the administered rAAV comprising PAH results in the increase of tyrosine (Tyr) in the subject. Without wishing to be bound by theory, various modes of action may result in the increase of Tyr in the subject including, for example, increased production of Tyr, increased transport or trafficking of Tyr, and/or increased stability of Tyr. In some embodiments, the increase of Tyr is detected in plasma of the subject. In some embodiments, the increase of Tyr is detected in brain tissue of the subject. In some embodiments, the increase of Tyr is detected in liver tissue of the subject. In some embodiments, the administered rAAV comprising PAH increases Tyr in the subject by about 95%, 90%, 85%, 80%, 75%, 70%, 65%, 60%, 55%, 50%, 45%, 40%, 35%, 30%, 25%, 20%, 15%, or about 10% in comparison to the subject's baseline Tyr level prior to administering the rAAV comprising PAH. Accordingly, in some embodiments, the administered rAAV comprising PAH increases Tyr in the subject by about 95%. In some embodiments, the administered rAAV comprising PAH increases Tyr in the subject by about 90%. In some embodiments, the administered rAAV comprising PAH increases Tyr in the subject by about 85%. In some embodiments, the administered rAAV comprising PAH increases Tyr in the subject by about 80%. In some embodiments, the administered rAAV comprising PAH increases Tyr in the subject by about 75%. In some embodiments, the administered rAAV comprising PAH increases Tyr in the subject by about 70%. In some embodiments, the administered rAAV comprising PAH increases Tyr in the subject by about 65%. In some embodiments, the administered rAAV comprising PAH increases Tyr in the subject by about 60%. In some embodiments, the administered rAAV comprising PAH increases Tyr in the subject by about 55%. In some embodiments, the administered rAAV comprising PAH increases Tyr in the subject by about 50%. In some embodiments, the administered rAAV comprising PAH increases Tyr in the subject by about 45%. In some embodiments, the administered rAAV comprising PAH increases Tyr in the subject by about 40%. In some embodiments, the administered rAAV comprising PAH increases Tyr in the subject by about 35%. In some embodiments, the administered rAAV comprising PAH increases Tyr in the subject by about 30%. In some embodiments, the administered rAAV comprising PAH increases Tyr in the subject by about 25%. In some embodiments, the administered rAAV comprising PAH increases Tyr in the subject by about 20%. In some embodiments, the administered rAAV comprising PAH increases Tyr in the subject by about 15%. In some embodiments, the administered rAAV comprising PAH increases Tyr in the subject by about 10%.

In some embodiments, the administered rAAV comprising PAH results in the increase of tryptophan (Trp) in the subject. Without wishing to be bound by theory, various modes of action may result in the increase of Trp in the subject including, for example, increased production of Trp, increased transport or trafficking of Trp, and/or increased stability of Trp. In some embodiments, the increase of Trp is detected in plasma of the subject. In some embodiments, the increase of Trp is detected in brain tissue of the subject. In some embodiments, the increase of Trp is detected in liver tissue of the subject. In some embodiments, the administered rAAV comprising PAH increases Trp in the subject by about 95%, 90%, 85%, 80%, 75%, 70%, 65%, 60%, 55%, 50%, 45%, 40%, 35%, 30%, 25%, 20%, 15%, or about 10% in comparison to the subject's baseline Trp level prior to administering the rAAV comprising PAH. Accordingly, in some embodiments, the administered rAAV comprising PAH increases Trp in the subject by about 95%. In some embodiments, the administered rAAV comprising PAH increases Trp in the subject by about 90%. In some embodiments, the administered rAAV comprising PAH increases Trp in the subject by about 85%. In some embodiments, the administered rAAV comprising PAH increases Trp in the subject by about 80%. In some embodiments, the administered rAAV comprising PAH increases Trp in the subject by about 75%. In some embodiments, the administered rAAV comprising PAH increases Trp in the subject by about 70%. In some embodiments, the administered rAAV comprising PAH increases Trp in the subject by about 65%. In some embodiments, the administered rAAV comprising PAH increases Trp in the subject by about 60%. In some embodiments, the administered rAAV comprising PAH increases Trp in the subject by about 55%. In some embodiments, the administered rAAV comprising PAH increases Trp in the subject by about 50%. In some embodiments, the administered rAAV comprising PAH increases Trp in the subject by about 45%. In some embodiments, the administered rAAV comprising PAH increases Trp in the subject by about 40%. In some embodiments, the administered rAAV comprising PAH increases Trp in the subject by about 35%. In some embodiments, the administered rAAV comprising PAH increases Trp in the subject by about 30%. In some embodiments, the administered rAAV comprising PAH increases Trp in the subject by about 25%. In some embodiments, the administered rAAV comprising PAH increases Trp in the subject by about 20%. In some embodiments, the administered rAAV comprising PAH increases Trp in the subject by about 15%. In some embodiments, the administered rAAV comprising PAH increases Trp in the subject by about 10%.

In some embodiments, the administered rAAV comprising PAH results in the increase of neurotransmitters in the subject. Without wishing to be bound by theory, various modes of action may result in the increase of neurotransmitters in the subject including, for example, increased production of neurotransmitters, increased transport or trafficking of neurotransmitters, and/or increased stability of neurotransmitters. In some embodiments, the increase of neurotransmitters is detected in brain tissue of the subject. In some embodiments, the increase of neurotransmitters is detected in central nervous system (CNS) of the subject. In some embodiments, the neurotransmitter is serotonin, dopamine, noradrenaline, epinephrine, or norepinephrine. In some embodiments, the administered rAAV comprising PAH increases one or more neurotransmitters in the subject by about 95%, 90%, 85%, 80%, 75%, 70%, 65%, 60%, 55%, 50%, 45%, 40%, 35%, 30%, 25%, 20%, 15%, or about 10% in comparison to the subject's neurotransmitter baseline level prior to administering the rAAV comprising PAH. Accordingly, in some embodiments, the administered rAAV comprising PAH increases one or more neurotransmitters in the subject by about 95%. In some embodiments, the administered rAAV comprising PAH increases one or more neurotransmitters in the subject by about 90%. In some embodiments, the administered rAAV comprising PAH increases one or more neurotransmitters in the subject by about 85%. In some embodiments, the administered rAAV comprising PAH increases one or more neurotransmitters in the subject by about 80%. In some embodiments, the administered rAAV comprising PAH increases one or more neurotransmitters in the subject by about 75%. In some embodiments, the administered rAAV comprising PAH increases one or more neurotransmitters in the subject by about 70%. In some embodiments, the administered rAAV comprising PAH increases one or more neurotransmitters in the subject by about 65%. In some embodiments, the administered rAAV comprising PAH increases one or more neurotransmitters in the subject by about 60%. In some embodiments, the administered rAAV comprising PAH increases one or more neurotransmitters in the subject by about 55%. In some embodiments, the administered rAAV comprising PAH increases one or more neurotransmitters in the subject by about 50%. In some embodiments, the administered rAAV comprising PAH increases one or more neurotransmitters in the subject by about 45%. In some embodiments, the administered rAAV comprising PAH increases one or more neurotransmitters in the subject by about 40%. In some embodiments, the administered rAAV comprising PAH increases one or more neurotransmitters in the subject by about 35%. In some embodiments, the administered rAAV comprising PAH increases one or more neurotransmitters in the subject by about 30%. In some embodiments, the administered rAAV comprising PAH increases one or more neurotransmitters in the subject by about 25%. In some embodiments, the administered rAAV comprising PAH increases one or more neurotransmitters in the subject by about 20%. In some embodiments, the administered rAAV comprising PAH increases one or more neurotransmitters in the subject by about 15%. In some embodiments, the administered rAAV comprising PAH increases one or more neurotransmitters in the subject by about 10%.

In some embodiments, following administration of the AAV vector to the subject the levels of functional PAH detectable in the subject are between about 2 and 10 times greater than the amount of functional PAH detectable in the subject before administration of the rAAV comprising PAH.

In some embodiments, following administration of the AAV vector to the subject the levels of detectable functional PAH meets or exceeds the human therapeutic level. In some embodiments, the levels of functional PAH post administration of the rAAV vector is about between 2 and 35 times the human therapeutic level. In some embodiments, the levels of active PAH post administration is about 2 times the human therapeutic level. In some embodiments, the levels of functional PAH post administration is about 3 times the human therapeutic level. In some embodiments, the levels of functional PAH post administration is about 4 times the human therapeutic level. In some embodiments, the levels of active PAH post administration is about 5 times the human therapeutic level. In some embodiments, the levels of active PAH post administration is about 6 times the human therapeutic level. In some embodiments, the levels of active PAH post administration is about 6 times the human therapeutic level. In some embodiments, the levels of active PAH post administration is about 7 times the human therapeutic level. In some embodiments, the levels of active PAH post administration is about 8 times the human therapeutic level. In some embodiments, the levels of active PAH post administration is about 9 times the human therapeutic level. In some embodiments, the levels of active PAH post administration is about 10 times the human therapeutic level. In some embodiments, the levels of active PAH post administration is about 15 times the human therapeutic level. In some embodiments, the levels of active PAH post administration is about 20 times the human therapeutic level. In some embodiments, the levels of active PAH post administration is about 25 times the human therapeutic level. In some embodiments, the levels of active PAH post administration is about 30 times the human therapeutic level. In some embodiments, the levels of functional PAH post administration is about 35 times the human therapeutic level.

In some embodiments, the rAAV PAH vector is delivered as a single dose per subject. In some embodiments, the subject is delivered the minimal effective dose (MED). As used herein, MED refers to the rAAV PAH vector dose required to achieve PAH activity resulting in reduced Phe levels in a subject.

The vector titer is determined on the basis of the DNA content of the vector preparation. In some embodiments, quantitative PCR or optimized quantitative PCR is used to determine the DNA content of the rAAV PAH vector preparations. Examples of optimized quantitative PCR include double droplet PCR. In one embodiment, the dosage is about 1×10¹¹ vector genomes (vg)/kg body weight to about 1×10¹³ vg/kg, inclusive of endpoints.

In some embodiments, the dosage is 1×10ⁿ vg/kg. In another embodiment, the dosage is 1×10¹² vg/kg. In specific embodiments, the dose of rAAV.hPAH administered to a subject is at least 1×10¹⁰ vg/kg, 5×10¹⁰ vg/kg, 1×10¹¹ vg/kg, 5.0×10¹¹ vg/kg, 1×10¹² vg/kg, 2.0×10¹² vg/kg, 3.5×10¹² vg/kg, 4.0×10¹² vg/kg, 4.5×10¹² vg/kg, 5.0×10¹² vg/kg, 5.5×10¹² vg/kg, 6.0×10¹² vg/kg, 6.5×10¹² vg/kg, 7.0×10¹² vg/kg, 8.0×10¹² vg/kg, 9.0×10¹² vg/kg, 1.0×10¹³ vg/kg, 2.5×10¹³ vg/kg, 5×10¹³ vg/kg, 7.5×10¹³ vg/kg, or 1.0×10¹⁴ vg/kg.

In some embodiments, the rAAV PAH vector compositions can be formulated in dosage units to contain an amount of replication-defective virus that is in the range of about 1.0×10⁹ vg to about 1.0×10¹⁵ vg. As used herein, the term “dosage” can refer to the total dosage delivered to the subject in the course of treatment, or the amount delivered in a single round of administration in the course of treatment comprising multiple rounds of administration.

In some embodiments, the dosage is sufficient to decrease plasma Phe levels in the patient by 25% or more. In some embodiments, rAAV PAH is administered in combination with one or more therapies for the treatment of PKU. In some embodiments, rAAV PAH is administered in combination with a PKU diet. In some embodiments, rAAV PAH is administered in combination with a low-protein diet. In some embodiments, rAAV PAH is administered in combination with a PKU nutraceutical or nutritional supplement or nutritional formula. In some embodiments, rAAV PAH is administered in combination with a neutral amino acid therapy. In some embodiments, rAAV PAH is administered in combination with pharmacologic drugs. In some embodiments, rAAV PAH is administered with sapropterin dihydrochloride. In some embodiments, rAAV PAH is administered in combination with Kuvan®. In some embodiments, rAAV PAH is administered in combination with PKU metabolizing enzymes. In some embodiments, rAAV PAH is administered in combination with pegavaliase. In some embodiments, rAAV PAH is administered in combination with Palynziq®. In some embodiments, the rAAV administration precedes other PKU therapies, is concomitant with or is delivered post-administration of other PKU therapies.

### EXAMPLES

Other features, objects, and advantages of the present invention are apparent in the examples that follow. It should be understood, however, that the examples, while indicating embodiments of the present invention, are given by way of illustration only, not limitation. Various changes and modifications within the scope of the invention will become apparent to those skilled in the art from the examples.

### Example 1. Vector Design

Exemplary methods and designs of generating representative rAAV expression constructs (rAAV vectors) comprising phenylalanine hydroxylase (PAH) sequences and variations of the same are provided in this Example.

In this study, recombinant AAV vector (rAAV8) was used. The basic design of a rAAV vector comprises an expression cassette flanked by inverted terminal repeats (ITRs): a 5′-ITR and a 3′-ITR. These ITRs mediate the replication and packaging of the vector genome by the AAV replication protein Rep and associated factors in vector producer cells. Typically, an expression cassette contains a promoter, a coding sequence, a polyA tail and/or a tag, as shown in FIG. 1A and FIG. 1B. An exemplary expression construct encoding hPAH was designed and prepared using standard molecular biology techniques. The coding sequence for the hPAH was inserted downstream of a promoter, hTTR (human transthyrethin promoter). Additionally, liver-specific cis-acting regulatory module (CRM) was inserted upstream of the promoter, and a minute virus of mice (MVM) intron sequence was inserted downstream of the promoter. This regulatory and promoter combination was tested to assess transduction levels, as shown in the examples that follow. The expression construct was subsequently ligated to the AAV vector and verified by sequencing.

Codon Optimization

The coding sequences for the hPAH were codon-optimized based on multiple parameters, such as CpG site count, GC content, palindromes, repetitious base sequences and exclusion of restriction sites and splice cites. The number of CpG island sequences, which can elicit immune response, were reduced to less than 6. The GC content was maintained approximately at 57% (±3%). Repetitious bases, which were greater or equal to 10 bp were also removed.

A schematic for exemplary constructs comprising PAH are shown in FIGS. 1A-1D. Any number of variations of the above construct can be performed. For example, more than one promoter may be used, and/or a WPRE sequence may be introduced. Additionally, different combinations of regulatory region, promoter, and intron are contemplated.

### Example 2. In Vitro Expression of rAAV8 Vectors Comprising Codon-Optimized hPAH Sequence

This example illustrates that rAAV8 vectors comprising codon-optimized hPAH sequence are effective in inducing expression of hPAH in vitro.

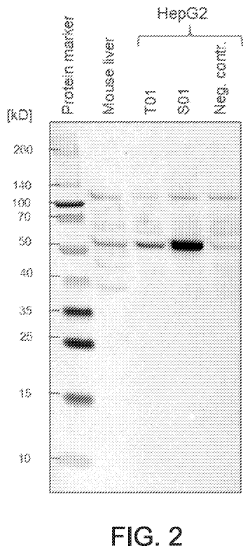

HepG2 (human liver cancer cell line) cells were infected by rAAV vectors comprising either a wild-type hPAH sequence or a codon-optimized hPAH sequence. The level of PAH expression in the cell lysates was measured using Western blot with an antibody against PAH. As shown in FIG. 2, rAAV8 comprising a codon-optimized hPAH sequence (S01) resulted in greater expression of hPAH compared to the rAAV8 comprising the wild-type hPAH (T01).

### Example 3. In Vivo Efficacy of rAAV8 Vectors with Codon-Optimized hPAH Sequence

This example illustrates the in vivo efficacy of the codon-optimized rAAV8 hPAH constructs in normalizing plasma levels of Phe, Trp and Tyr in PAH knock-out (PAH-KO) mice.

PAH-KO mice were injected with rAAV vectors comprising a wild-type hPAH sequence (Group A, T01); or codon-optimized hPAH sequences (Group B and C, S01 and S03). The rAAV vector constructs are depicted in FIG. 1A and FIG. 1B. Mice received 1×10¹³ vg/kg of vectors and plasma samples were collected prior to administration of the rAAV and at week 1, week 2, week 3, week 4, and week 5 post injection. Mice were sacrificed at week 5, and tissue samples were harvested. Additionally, coat color of the mice was monitored. A group of wild-type mice and a group of untreated PAH-KO mice were used as positive and negative controls, respectively. The experimental design is summarized in Table 3, below.

TABLE 3
In vivo study using rAAV8 vectors that comprise
wild-type and codon-optimized PAH

| Group | Condition | Treatment | Dose |
|---|---|---|---|
| A | Control vector | T01 | 1 × 10¹³ vg/kg |
| B | Test | S01 | 1 × 10¹³ vg/kg |
| C | Test | S03 | 1 × 10¹³ vg/kg |
| D | Positive Control | WT; Untreated | — |
| E | Negative Control | PAH-KO; Untreated | — |

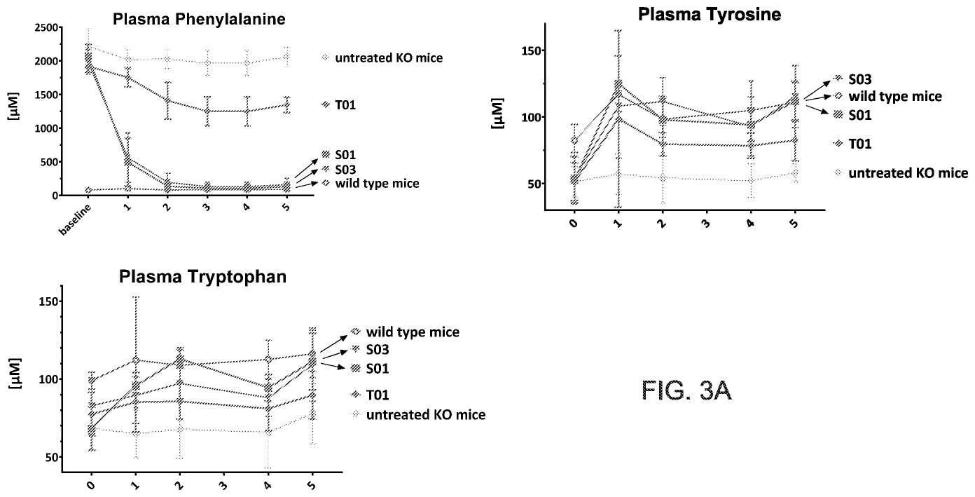

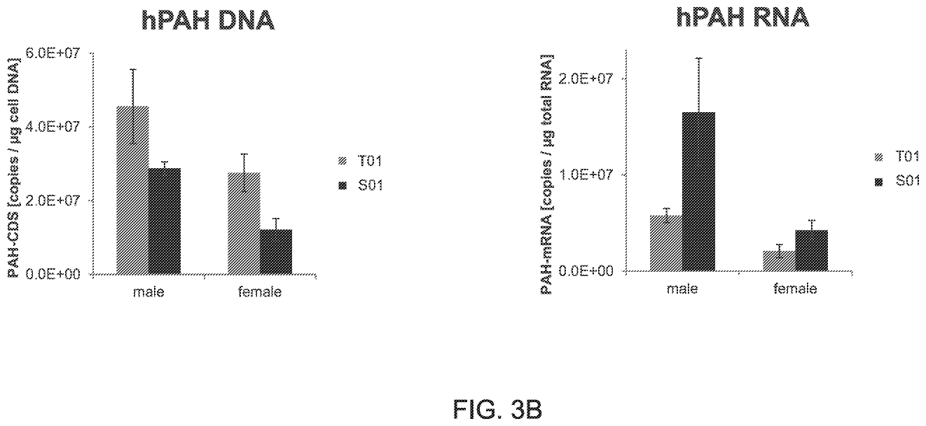

The efficacy of vector-mediated expressed PAH was determined by monitoring the plasma levels of phenylalanine (Phe), tyrosine, (Try) and tryptophan (Trp). The PAH enzyme is responsible for the first step in processing phenylalanine and involved in biosynthesis of Tyr and Trp. Results are depicted in FIG. 3A. Mice administered with the codon-optimized constructs, S01 (Group B) and S03 (Group C) showed significantly reduced Phe concentration in plasma compared to untreated mice or mice administered with the control vector, T01 (Group A). After 2 weeks post administration, the level of Phe in mice of Group B and C was similar to that of the wild type mice (Group D). Moreover, the decreased level of Phe was maintained after 5 weeks following a single dose administration. The level of Tyr and Trp also increased in mice of Group B and C compared to that of Group A or untreated KO mice (Group E).

Furthermore, the transcription efficiency of the rAAV8 comprising the codon-optimized human PAH was compared to the transcription efficiency of the rAAV8 comprising wild type human PAH. The results are presented in FIG. 3B and show that while there was comparable transduction between the codon-optimized human PAH comprising rAAV8 and the wild type human PAH comprising rAAV8, there was superior transcription of codon-optimized hPAH.

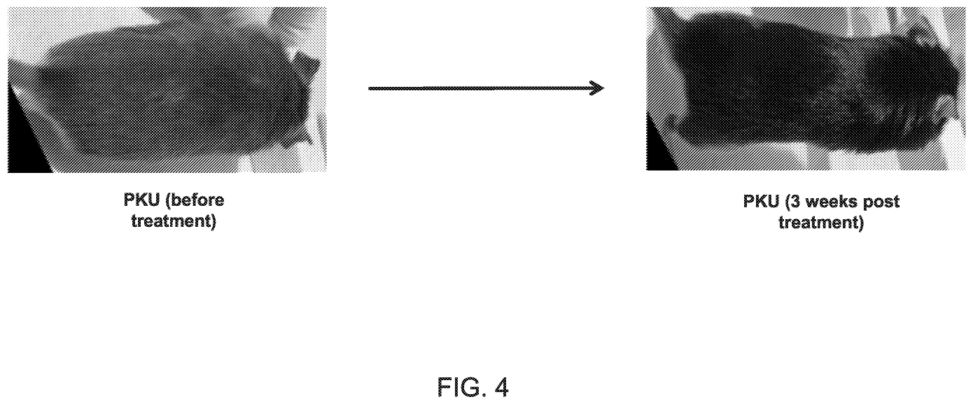

The coat color of mice was also monitored. Surprisingly, the coat color was corrected at week 3 post administration of rAAV8 with codon-optimized PAH, as shown in FIG. 4.

### Example 4. Gene Therapy Using rAAV8 Vectors Comprising Codon-Optimized hPAH Normalizes PKU Biomarkers in a Dose-Dependent Manner

This example illustrates that gene therapy using the rAAV8 vectors comprising codon-optimized hPAH sequence normalizes the plasma levels of Phe, Tyr and Trp and coat color in a dose-dependent manner.

PAH-KOmice were injected with rAAV8 vectors expressing codon-optimized hPAH vector at low (1×10¹² vg/kg) or high (1×10¹³ vg/kg) dose. Plasma samples were collected prior to administration of the rAAV and at week 1, week 2, week 3, week 4, and week 5 post injection and the levels of Phe, Tyr and Trp were measured at each time point. Additionally coat color of mice was monitored. A group of untreated PAH-KO mice were used as a negative control. The experimental design is summarized in Table 4, below.

TABLE 4
Dose-dependent effect of gene therapy using rAAV8
vectors with wild-type codon-optimized PAH

| | | | | Number of |
|---|---|---|---|---|
| Group | Condition | Treatment | Dose | Mice (N) |
| F | Low dose | S01 | 1 × 10¹² vg/kg | 6 |
| G | High dose | S01 | 1 × 10¹³ vg/kg | 6 |
| H | Negative | PAH-KO; Untreated | — | 6 |
| | Control | | | |

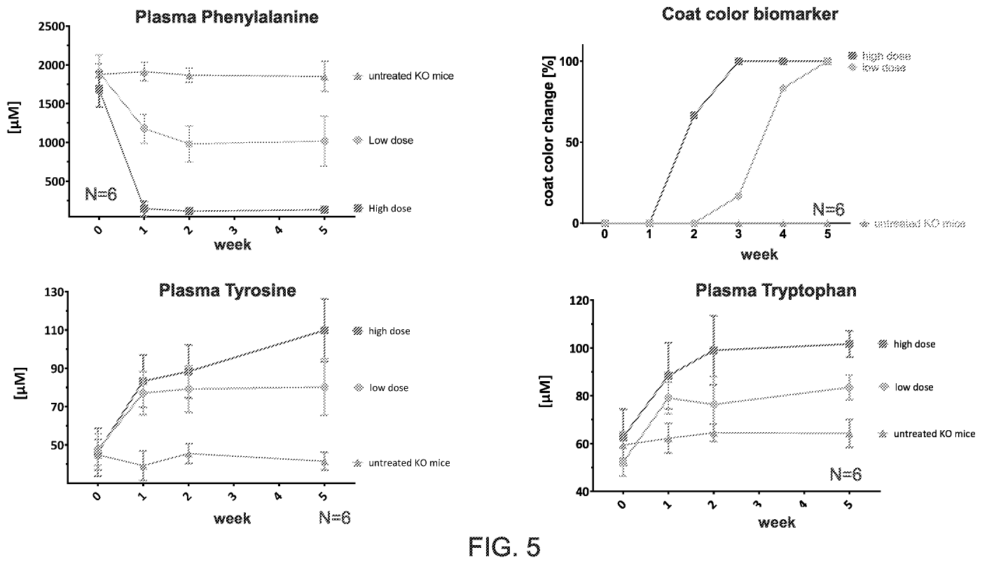

Results are depicted in FIG. 5. Plasma Phe level was significantly decreased in mice administered with the codon-optimized hPAH in a dose-dependent manner. Mice administered with high dose of S01 showed 100% coat color conversion after three weeks. Low dose, which is 10-fold lower than the high dose, shows clinical benefit with delayed kinetics. Additionally, tyrosine and tryptophan in plasma of PAH-KO mice were normalized following low dose administration of S01.

### Example 5. Normalization of Neutral Amino Acid and Neurotransmitter Levels in Brain of PAH-KO Mice by Gene Therapy with AAV8 Vectors Comprising Codon-Optimized hPAH

This example illustrates in vivo efficacy of the codon-optimized rAAV8 hPAH constructs in normalizing the levels of Phe, Trp and Tyr in the brain of PAH knock-out (PAH-KO) mice. Additionally, the levels of dopamine and serotonin in brains were restored in PAH-KO mice-treated by gene therapy with AAV8 vectors comprising codon-optimized hPAH sequence.

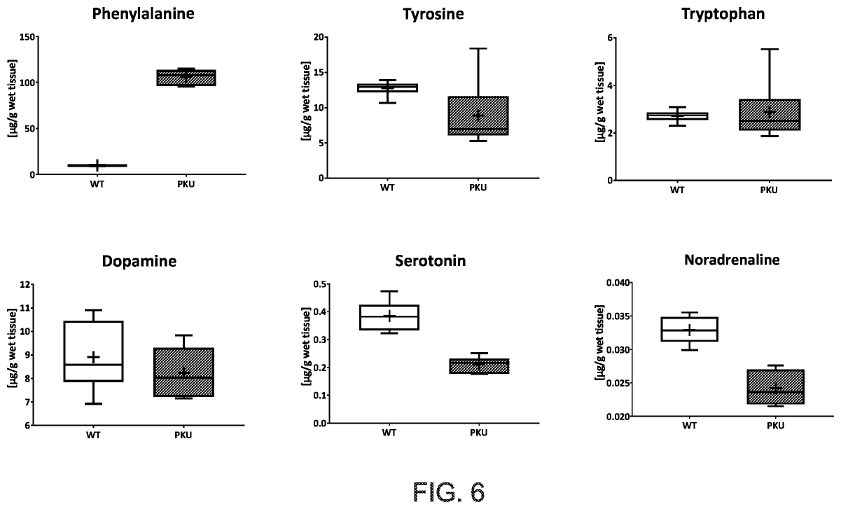

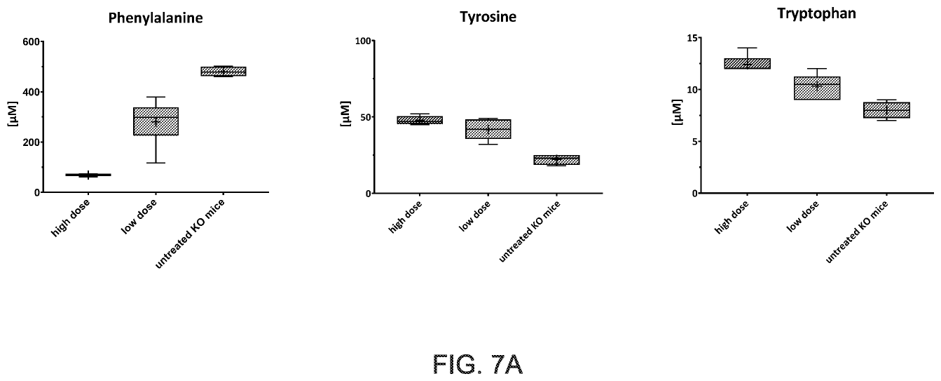

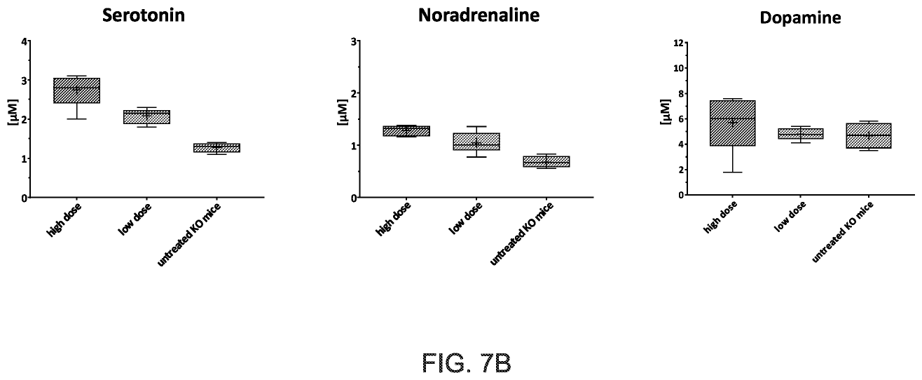

First, studies were performed to assess the levels of phenylalanine, tyrosine and tryptophan in brains of wild-type (wt) and PAH-KO mice. Among all the large neutral amino acids (LNAAs), phenylalanine has highest affinity for large neutral amino acid transporter (LNAAT), which transports LNAAs across the blood-brain barrier (BBB). If Phe is in excess in the blood, it saturates the transporter, and thus, decreases the levels of non-Phe LNAAs in the brain. As these amino acids are necessary for protein and neurotransmitter synthesis, Phe build-up hinders the development and functioning of the brain. Indeed, phenotyping of WT and PAH-KO mice confirmed that LNAAs (Phe, Try, and Trp) and neurotransmitters (dopamine, serotonin, and noradrenaline) are dysregulated in brains of PAH-KO mice, as shown in FIG. 6. Concentrations of Phe, Tyr, and Trp were measured in brain tissues extracted at week 5 from the mouse groups shown in Table 4. The results are shown in FIG. 7A. Levels of Phe in brain significantly decreased in PAH-KO mice treated by gene therapy with AAV8 with codon-optimized hPAH sequence in a dose-dependent manner. Consistently, the levels of both Tyr and Trp were increased in brains of PAH-KO mice treated by gene therapy with AAV8 comprising codon-optimized hPAH sequence in a dose-dependent manner. Moreover, both serotonin and noradrenaline levels were restored in brains of treated PAH-KO mice in a dose-dependent manner, as shown in FIG. 7B.

### Example 6. Long-Term Stabilization of Phe Levels in PAH-KO Mice by Gene Therapy with AAV8 Vectors Comprising Codon-Optimized hPAH

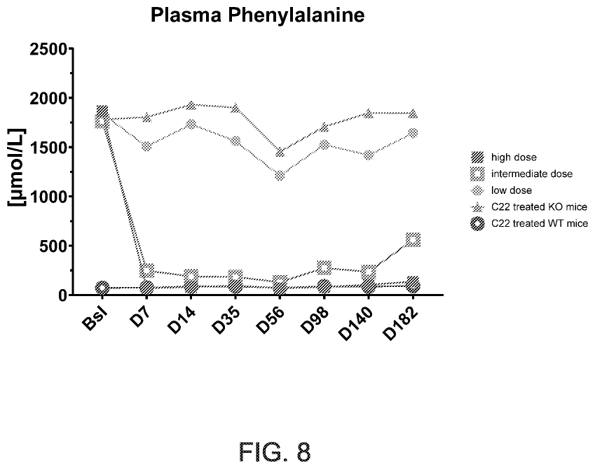

This example illustrates long-term in vivo efficacy of gene therapy with AAV8 vectors comprising codon-optimized hPAH sequence in PAH-KO mice (FIG. 8).

PAH knock-out (PAH-KO) mice were injected with codon-optimized rAAV8 hPAH constructs at low (1×10¹² vg/kg), intermediate or high doses (1×10¹³ vg/kg). Plasma samples were collected prior to administration of the rAAV8 and at 7, 14, 35, 56, 98, 140 and 182 days post injection. Levels of phenylalanine were measured in plasma and compared to levels of Phe in control (C22)-treated PAH-KO mice and control C22-treated wt mice. The results are shown in FIG. 8.

Levels of Phe were significantly decreased in PAH-KO mice treated with codon-optimized rAAV8 hPAH constructs relative to control C22-treated PAH-KO mice. The Phe levels in mice that received hPAH treatment were comparable to Phe levels in C22 control-treated wt mice.

At a high dose of 1×10¹³ vg/kg, plasma Phe levels were sustained at low levels similar to baseline levels and comparable to Phe levels in wt mice at 7, 14, 35, 56, 98, 140 and 182 days post-administration demonstrating the long-term efficacy of gene therapy with AAV8 codon-optimized hPAH in PAH-KO mice.

### EQUIVALENTS AND SCOPE

Those skilled in the art will recognize, or be able to ascertain using no more than routine experimentation, many equivalents to the specific embodiments of the invention described herein. The scope of the present invention is not intended to be limited to the above description, but rather is as set forth in the following claims:

We claim:

1. A recombinant adeno-associated virus vector (rAAV) comprising a codon-optimized sequence encoding a human phenylalanine hydroxylase (PAH), wherein the codon-optimized sequence has at least 70% identity to one of SEQ ID Nos: 11-27.

2. The rAAV of claim 1, wherein the codon-optimized sequence has at least 75%, 80%, 85%, 90%, 95% or 99% identity to one of SEQ ID Nos: 11-27.

3. The rAAV of claim 1 or 2, wherein the codon-optimized sequence is identical to one of SEQ ID Nos: 11-27.

4. The rAAV of any one of the preceding claims, wherein the rAAV encodes an AAV8 capsid.

5. The rAAV of claim 4, wherein the AAV8 capsid is a modified AAV8 capsid with improved liver tropism compared to the wild-type AAV8 capsid.

6. The rAAV of claim 5, wherein the AAV8 capsid has at least 70%, 75%, 80%, 85%, 90%, 95% or 99% identity to the wild-type AAV8 capsid.

7. The rAAV of any one of the preceding claims, wherein the rAAV further comprises a WPRE sequence.

8. The rAAV of claim 7, wherein the WPRE sequence is a naturally-occurring WPRE sequence.

9. The rAAV of claim 7, wherein the WPRE sequence is a modified WPRE sequence.

10. The rAAV of claim of any one of the preceding claims, wherein the rAAV further comprises a liver-specific promoter.

11. The rAAV of claim 10, wherein the liver-specific promoter is a transthyretin promotor (TTR).

12. The rAAV of any one of the preceding claims, wherein the rAAV comprises a cis-acting regulatory module (CRM).

13. The rAAV of claim 12, wherein the vector comprises one, two, three, four, five or more CRM repeats.

14. The rAAV of claim 12 or 13, wherein the CRM is CRM8.

15. The rAAV of any one of the preceding claims, wherein the rAAV further comprises an intron upstream of the PAH sequence.

16. The rAAV of claim 15, wherein the intron is a minute virus of mice (MVM) intron.

17. A method of treating phenylketonuria (PKU), comprising administering to a subject in need of treatment a recombinant adeno-associated virus (rAAV) comprising a codon-optimized sequence encoding a human phenylalanine hydroxylase (PAH), wherein the codon-optimized sequence has at least 70% identity to one of SEQ ID Nos: 11-27.

18. The method of claim 17, wherein administering the rAAV results in a decrease in plasma phenylalanine (Phe) level in the subject compared to a control.

19. The method of claim 17 or 18, wherein administering the rAAV results in an increase in plasma tyrosine level in the subject compared to a control.

20. The method of any one of claims 17-19, wherein administering the rAAV results in an increase in plasma tryptophan level in the subject compared to a control.

21. The method of any one of claims 18-20, wherein the control is the pre-treatment level in the subject.

22. The method of any one of claims 18-20, wherein the control is a reference level based on historical data.

23. The method of any one of claims 17-22, wherein the codon-optimized sequence has at least 75%, 80%, 85%, 90%, 95% or 99% identity to one of SEQ ID Nos: 11-27.

24. The method of any one of claims 17-23, wherein the codon-optimized sequence is identical to one of SEQ ID Nos: 11-27.

25. The method of any one of claims 17-24, wherein the rAAV encodes an AAV8 capsid.

26. The method of claim 25, wherein the AAV8 capsid is a modified AAV8 capsid with improved liver tropism compared to the wild-type AAV8 capsid.

27. The method of claim 25, wherein the AAV8 capsid has at least 70%, 75%, 80%, 85%, 90%, 95% or 99% identity to the wild-type AAV capsid.

28. The method of any one of claims 17-27, wherein the rAAV further comprises a WPRE sequence.

29. The method of claim 28, wherein the WPRE sequence is a naturally-occurring WPRE sequence.

30. The method of claim 28, wherein the WPRE sequence is a modified WPRE sequence.

31. The method of any one of claims 17-30, wherein the rAAV further comprises a liver-specific promoter.

32. The method of claim 31, wherein the liver-specific promoter is a transthyretin promotor (TTR).

33. The method of any one of claims 17-32, wherein the rAAV comprises a cis-acting regulatory module (CRM).

34. The method of claim 33, wherein the vector comprises one, two, three, four, five or more CRM repeats.

35. The method of claim 33 or 34, wherein the CRM is CRM8.

36. The method of any one of claims 17-35, wherein the rAAV further comprises an intron upstream of the PAH sequence.

37. The method of claim 36, wherein the intron is a minute virus of mice (MVM) intron.

38. The method of any one of claims 17-37, wherein the rAAV is administered at dose of about 1×10¹⁰ vg/kg, about 1×10¹¹ vg/kg, about 1×10¹² vg/kg, about 1×10¹³ vg/kg, about 1×10¹⁴ vg/kg, or about 1×10¹⁵ vg/kg.

39. The method of any one of claims 17-38, wherein the rAAV is administered systemically.

40. The method of any one of claims 17-38, wherein the rAAV is administered intravenously.
